(19) United States
(12) Patent Application Publication
(10) Pub. No.: US 2019/0365653 A1
(43) Pub. Date: Dec. 5, 2019

(54) NOVEL RECOMBINANT EXOSOME CONTAINING HYALURONIDASE AND USE THEREOF

(71) Applicant: KOREA INSTITUTE OF SCIENCE AND TECHNOLOGY, Seoul (KR)

(72) Inventors: Yeon-Sun Hong, Seoul (KR); Yoo Soo Yang, Seoul (KR); In-San Kim, Seoul (KR)

(73) Assignee: KOREA INSTITUTE OF SCIENCE AND TECHNOLOGY, Seoul (KR)

(21) Appl. No.: 15/996,729

(22) Filed: Jun. 4, 2018

(51) Int. Cl. A61K 9/48; A61K 38/47; A61K 31/407; A61K 31/337; A61K 39/395; A61P 35/00; A61K 41/00; A61K 31/704; A61K 45/06

(52) U.S. Cl. CPC A61K 9/48; C12Y 302/01035; A61K 38/47; A61K 31/407; A61K 31/337; A61N 5/10; A61P 35/00; A61K 41/0057; A61K 31/704; A61K 45/06; A61K 39/3955

(57) ABSTRACT

The present invention relates to a recombinant exosome containing hyaluronidase and a use thereof, and more particularly to a recombinant exosome that presents hyaluronidase on its surface and use thereof as an anticancer agent.

### SEQUENCE LISTING

The instant application contains a Sequence Listing which has been submitted electronically in ASCII format and is hereby incorporated by reference in its entirety. Said ASCII copy, created on Aug. 27, 2018, is named 115518-0106_SL.txt and is 108 KB in size.

### TECHNICAL FIELD

The present invention relates to a novel recombinant exosome and use thereof, and more particularly to a novel recombinant exosome which presents hyaluronidase on its surface and use thereof.

### BACKGROUND OF THE INVENTION

Despite recent progress of the research on nanomedicine for cancer treatment, most nanoparticles used for drug delivery have limited their therapeutic efficacy because they do not penetrate deeply into tumor tissue and are localized only around the vessels of solid tumors (Manzoor et al., Cancer Res., 72: 5566-5575, 2012; Jain and Stylianopoulos, Nat. Rev. Clin. Oncol. 7: 653-664, 2010; and Minchinton and Tannock, Nat. Rev. Cancer 6: 583-592, 2006). The degree of accumulation of nanoparticles in the tumor may be severely limited by the elevation of interstitial fluid pressure and the dense extracellular matrix (ECM) complex.

The ECM is composed primarily of hyaluronic acid (HA) and dense collagen networks embedded in a gel of gluosaminoglycans (Toole, Nat. Rev. Cancer, 4(7): 528-539, 2004; Evanko et al., Adv. Drug Deliv. Rev., 59(13): 1351-1365, 2007), creating a formidable physical barrier and a hydrodynamic barrier in the form of interstitial pressure in the tumor microenvironment (Shepard, Front. Oncol., 5: 192, 2015; Heldin et al., Nat. Rev. Cancer 4 (10): 806-813, 2004; Waite et al., Crit. Rev. Biomed. Eng., 40(1): 21-41, 2012).

Therapeutic strategies aimed at disrupting the ECM within the tumor microenvironment have been developed rapidly (Whatcott et al., Cancer Discov. 1(4): 291-296, 2011; Tong and Kohane, Annu. Rev. Pharmacol., 56: 41-57, 2016) through several clinical trials involving PEG-PH20 (PEGylated rHuPH20 enzyme, recombinant human hyaluronidase enzyme) (Thompson et al., Mol. Cancer Ther., 9(11): 3052-3064, 2011; Zhou et al., Nano Lett., 16(5): 3268-3277, 2016; Hingorani et al., Clin. Cancer Res., 22(12): 2848-2854, 2016). Also, the multivalent presentation of ECM degrading enzymes in polymeric nanoparticles has been developed in order to enhance the efficiency of enzyme delivery and the diffusion of nanoparticles in tumors (Villegas et al., ACS Appl. Mater. Interfaces 7(43): 24075-24081, 2015; Goodman et al., Int. J. Nanomedicine, 2(2): 265-274, 2007; Wong et al., Proc. Natl. Acad. Sci. USA 108(6): 2426-2431; 2011; Zhou et al., Theranostics 6(7): 1012-1022, 2016). However, a possible problem with this nanoformulation is that the properties of the enzyme could be irreversibly changed by the immobilization process. Moreover, there is some concern regarding the therapeutic efficacy of chemically modified synthetic nanoparticles displaying truncated forms of PH20 lacking the membrane-bound glycosyl phosphatidylinositol (GPI) domain (Rosengren et al., AAPS J. 17 5: 1144-1156, 2015; Arming et al., Eur. J. Biochem., 247 (3): 810-814, 1997). The GPI anchor of native PH20 hyaluronidase has been shown to maximize the successful penetration of sperm into the egg vestments and facilitate the fusion of egg-sperm plasma membranes by increasing the mobility of the protein on cell surface and facilitating the fusion of egg-sperm plasm membranes (Hunnicutt et al., Biol. Reprod., 54(6): 1343-1349, 1996; Sauber et al., J. Androl., 18(2): 151-158, 1997).

Accordingly, the present invention has been made to solve the above-mentioned problems, and it is an object of the present invention to provide a more effective nanodrug based on PH20 and an anticancer immunotherapeutic agent using the same. However, these problems are exemplary and do not limit the scope of the present invention.

### SUMMARY OF THE INVENTION

According to one aspect of the present invention, there is provided a recombinant exosome that presents a hyaluronidase on its surface.

According to another aspect of the present invention, there is provided a composition for treating cancer comprising the recombinant exosome as an active ingredient.

According to another aspect of the present invention, there is provided a composition for treating cancer comprising a recombinant exosome presenting a hyaluronidase on its surface and encapsulating an anticancer compound therein as an active ingredient.

According to another aspect of the present invention, there is provided a composition for treating cancer comprising a recombinant exosome that presents a hyaluronidase on its surface and an anticancer compound as an active ingredient.

According to another aspect of the present invention, there is provided a method for treating cancer in a subject, comprising administering a therapeutically effective amount of the recombinant exosome to a subject suffering from cancer.

According to another aspect of the present invention, there is provided a method for treating cancer, comprising administering a therapeutically effective amount of a recombinant exosome presenting a hyaluronidase on its surface and encapsulating an anticancer drug therein to a subject suffering cancer.

According to another aspect of the present invention, there is provided a method for treating cancer, comprising administering a therapeutically effective amount of a recombinant exosome that presents a hyaluronidase on its surface and a photodynamic therapy or a radiotherapy to a subject suffering from cancer.

### Effects of the Invention

The recombinant exosome according to one embodiment of the present invention as described above may be used for the treatment of cancer, since it can remove cancer cells selectively not depending on complex mechanisms such as gene delivery by degrading extracellular matrix that interferes with the access of immune cells and anticancer compounds by surrounding the tumor tissue.

### BRIEF DESCRIPTION OF DRAWINGS

FIGS. 1a to 1f show the preparation and characteristics of a recombinant exosome presenting PH20 on the surface (hereinafter abbreviated as “Exo-PH20”) according to an embodiment of the present invention derived from HEK293T cell line; FIG. 1a shows the results of western blot analysis using antibodies specific for PH20 and exosomal markers, Alix and Tsg101; FIG. 1b shows the results of western blot analysis using recombinant human PH20 (rHuPH20) diluted sequentially as a standard to quantify PH20 expression of Exo-PH20 of the present invention; FIG. 1c represents a graph showing the results of analysis of the size distribution of Exo-PH20 by dynamic light scattering analysis (DLS) according to an embodiment of the present invention, and a photograph of Exo-PH20 taken by transmission electron microscope (right side in the graph, scale bar: upper 100 nm and lower 20 nm); FIG. 1d represents a graph showing the size distribution of the control exosome (hereinafter abbreviated as “Exo-Con”) by dynamic light scattering analysis (DLS) and a photograph of Exo-Con taken by transmission electron microscope (right side in the graph, scale bar: upper 100 nm and lower 20 nm); FIG. 1e is a graph showing a comparison of enzymatic activity between the recombinant human PH20 and Exo-PH20 according to an embodiment of the present invention converted to the same mass; and FIG. if is a graph showing the results of western blot analysis to show that PH20 hyaluronidase expressed in Exo-PH20 presents in the lipid raft of exosomes.

FIGS. 2a to 2e show the preparation and characteristics of exosome in which PH20 is present on the surface and doxorubicin is embedded therein (hereinafter, ‘Exo-PH20^{Dox}’) according to one embodiment of the present invention; FIG. 2a is a graph showing the results of quantitation of doxorubicin loaded in 100 μg of exosomes reacted with varying amounts of doxorubicin (n=3 repeats per sample); FIG. 2b is a photograph of Exo-PH20^{Dox} taken by transmission electron microscope, wherein the scale bar on the left side shows 100 nm and the scale bar on the right side shows 20 nm, FIG. 2c is a graph showing the result of analysis of the size distribution of Exo-PH20^{Dox} by dynamic light scattering analysis (DLS); FIG. 2d is a graph showing the degree of doxorubicin release (%) over time in the physiological environment (pH 7.4, - -) and acidic conditions (pH 6.5, -▾-) similar to the cancer microenvironment; and FIG. 2e is a graph comparing enzymatic activity of recombinant PH20, Exo-PH20 and Exo-PH20^{Dox} converted to the same mass according to an embodiments of the present invention.

FIGS. 3a to 3c show the results of analysis of in vitro enzymatic ability of the PH20 surface-presenting recombinant exosome (Exo-PH20) according to an embodiment of the present invention; FIG. 3a represents a graph showing the results of measuring degree of tumor HA depletion after treating recombinant human PH20 (rHuPH20) and Exo-PH20 converted as the same mass by measuring relative lengths of experimental PC3 HA region compared with control PC3 HA region (left panel) and a series of images showing PC3 cells treated by recombinant human PH20 and Exo-PH20 converted as 53 ng of PH20 taken by a phase contrast microscope (scale bar: 100 μm); FIG. 3b is a series of images of PC3 cells treated with various amount (0, 5.3, 26.5, 53 ng) of recombinant human PH20 and Exo-PH20 converted as the same mass for 30 min which were taken by a phase contrast microscope, lower panels in each group represent enlarged images (scale bar: upper 50 μm and lower 100 μm); and FIG. 3c is a series of phase contrast microscopic images showing the result of real-time detection of tumor HA depletion treated with Exo-PH20 for 1 hour, wherein the HA (arrow) around HA-high expressing PC3 cancer cells was detected by excluding fixed erythrocytes.

FIGS. 4a to 4g show the results of analysis of in vivo anticancer activity after intratumoral administration of recombinant exosomes presenting PH20 on its surface (Exo-PH20) according to one embodiment of the present invention; FIG. 4a is a series of photographs showing the result of immunohistochemical analysis of tumor tissues extracted from PC3 tumor-bearing animals administered with Exo-PH20 (scale bar: 200 μm), wherein tumor sections obtained from tumor tissues of the PC3 tumor cell-xenograft animals treated with Exo-PH20 (5 mg/kg) for 6, 12, 24, 48, and 96 hours were stained with anti-PH20 antibody (top) and anti-HABP antibody (bottom); FIG. 4b shows the results of the immunohistochemical analysis of tumor tissues extracted from PC3-bearing animals administered with the same amount of Exo-Con as a control group, wherein tumor sections obtained from the tumor tissues of the PC3 tumor cell-xenograft animals treated with Exo-Con for 6, 12, 24, 48 and 96 hours were stained with anti-HABP antibody; FIG. 4c is a graph showing the result of measuring tumor volume over time after intratumoral administration of PBS, Exo-Con as a control, 530 ng of recombinant human PH20 (rHuPH20) and Exo-PH20 in amount corresponding to 530 ng of PH20, respectively to PC3 cell-bearing mouse model; FIG. 4d is a graph showing the body weight of the mouse over time after administration of PBS, Exo-Con as a control, 530 ng of recombinant human PH20 (rHuPH20) and Exo-PH20 in amount corresponding to 530 ng of PH20, respectively to PC3 cell-bearing mouse model; FIG. 4e is a graph representing weights of tumor tissues excised from PC3 cell-bearing cancer model mice administered intratumorally with 265 and 530 ng of recombinant human PH20 (rHuPH20), Exo-PH20 in amount corresponding to the dose of rHuPH20 (265 and 530 ng), Exo-Con as a control, and PBS, respectively (in the graph, the values represent mean±SD, *P<0.05, **P<0.01, and ***P<0.001 according to ANOVA analysis, and mice number of each group is 5); FIG. 4f is a series of images of PC3 tumor tissues excised 28 day after tumor transplantation from tumor-bearing mouse model administered with 265 and 530 ng of recombinant human PH20 (rHuPH20), Exo-PH20 corresponding to the dose of rHuPH20, Exo-Con as a control and PBS, respectively; and FIG. 4g is a series of images showing immunohistochemical analysis of tumor sections obtained 28 days after tumor transplantation in tumor-bearing mouse model administered with 530 ng of rHuPH20, Exo-PH20 in amount corresponding to the dose of rHuPH20, Exo-Con as a control and PBS, respectively, wherein the tumor sections were stained with anti-HABP antibody.

FIGS. 5a to 5g show the results of analysis of in vivo anticancer activity after intravenous injection of the recombinant exosome presenting PH20 on the surface (Exo-PH20) according to an embodiment of the present invention; FIG. 5a is a series of whole body fluorescent images showing in vivo distribution of exosomes over time (0, 1, 3, 6, 12, and 24 hours) after intravenous administration of Exo-Con, Exo-PH20 (10 mg/kg) labeled with cy5.5 fluorescent dye to BALB/c nude mice bearing PC3 tumor cells; FIG. 5b is a series of images of cancer tissue and organs (kidney, spleen, liver, heart and lung) showing the degree of accumulation of exosomes at 24 hours after intravenous administration of Exo-Con and Exo-PH20 (10 mg/kg) labeled with cy5.5 fluorescent dye to BALB/c nude mice having PC3 tumor cells; FIG. 5c is a graph showing tumor growth over time after intravenous administration of Exo-con, 10 mg/kg of Exo-PH20 to BALB/c nude mice bearing PC3 tumor cells (in the graph, the values represent mean±SD, *P<0.05, **P<0.01, and ***P<0.001 according to ANOVA analysis, and mice number of each group is 5); FIG. 5d is a graph showing the weight of tumors excised at the end of the experiment from the PC3-bearing mice of FIG. 5c (in the graph, the values represent mean±SD, *P<0.05, **P<0.01, and P<0.001 according to ANOVA analysis, and mice number of each group is 5); FIG. 5e is a series of images showing immunohistochemical analysis of PC3 tumor section obtained at the end of the experiment from the PC3-bearing mice of FIG. 5c using anti-HABP antibody; FIG. 5f represents the effect of Exo-PH20 on the microvessel region of PC3 tumors, the left panel is a graph showing relative blood flow (percent vascularization, %) over time (t=0, 1, 3, 6 and 24 hours) after intravenous administration of PBS (-●-), Exo-Con (-◯-, 10 mg/kg) and Exo-PH20 (-▾-, 10 mg/kg) to PC3-bearing mice by calculating blood vessel distribution per 20 mm² using high-resolution ultrasound imaging, and the right panel is a series of ultrasound images taken to check the state of blood flow at 24 hours after the administration; and FIG. 5g is a series of ultrasound images over time (t=0, 1, 3, 6 and 24 hours) after the administration in three groups.

FIG. 6 shows the results of analysis whether the penetration of nanoparticles into tumor tissues was increased after systemic administration of recombinant exosome presenting PH20 on the surface (Exo-PH20) according to one embodiment of the present invention and is a series of fluorescent images of tumor tissue sections obtained from PC3-bearing mice (the scale bar is 100 μm). The mice was administered with 10 mg/kg Exo-PH20 via intravenous injection through tail vein. After 3 hours, the administration of Exo-PH20 cy5.5-labeled liposome (4 mg/kg) was administered intravenously, and then after 7 hours, the injection of liposome the tumor tissues were excised, embedded in OCT medium and cryo-sectioned. The sections were analyzed under fluorescence microscope in order to observe the distribution of liposomes. Hoechst dye was used to stain the nucleus (blue), and blood vessels were stained with anti-CD31 antibody (green).

FIGS. 7a to 7d show the results of analysis of in vivo anticancer activity after systemic administration of the recombinant exosome presenting PH20 on the surface according to an embodiment of the present invention to a 4T1-bearing immune-competent mouse model (BALB/c); FIG. 7a is a graph showing tumor growth over time after systemic administration of PBS, 10 mg/kg of Exo-Con and 10 mg/kg of Exo-PH20, respectively (in the graph, the values represent mean±SD, *P<0.05, **P<0.01, and ***P<0.001 according to ANOVA analysis, and mice number of each group is 5); FIG. 7b. is a graph showing the results of measuring the weight of the tumor tissue after the end of the experiment of FIG. 7a (in the graph, the values represent mean±SD, *P<0.05, **P<0.01, and ***P<0.001 according to ANOVA analysis, and mice number of each group is 5); FIG. 7c is a series of immunofluorescent staining images showing CD8⁺ T cells infiltrated in cryo-sections of tumor tissues excised from the 4T1 orthotopic tumor model administered with PBS, 10 mg/kg of Exo-Con and 10 mg/kg of Exo-PH20 (scale bars represent 100 μm and after 25 days of tumor transplantation, excised tumor tissues were stained with anti-mouse CD8 antibody, and Fluor-488-labeled secondary antibody (green), Hoechst dye was used to stain the nucleus (blue)); FIG. 7d is a graph showing the number of CD8⁺ T cells infiltrated in cryo-sections of tumor tissues excised from the 4T1 orthotopic tumor models administered with PBS, 10 mg/kg of Exo-Con and 10 mg/kg of Exo-PH20 (Tumor invading CD8⁺ T cells were counted from 10 images containing the images shown in FIG. 7c using ImageJ software, and the values represent mean±SD, *P<0.05, **P<0.01, ***P<0.001 by ANOVA analysis).

FIGS. 8a to 8d show the results of analysis of the anticancer activity of recombinant exosome presenting PH20 on the surface and embedding doxorubicin therein according to an embodiment of the present invention; FIG. 8a is a graph showing the growth of tumors over time in PC3-bearing xenograft cancer model mice administered intravenously with PBS (-●-), free doxorubicin (free Dox, 1 mg/kg, - -), recombinant exosome presenting PH20 on the surface (Exo-PH20, mg/kg, —V—), control exosome embedding doxorubicin therein (Exo-Con^{Dox}, 10 mg/kg, corresponding to 1 mg/kg of doxorubicin, -Δ-), and recombinant exosome presenting PH20 on the surface embedding doxorubicin therein (Exo-PH20^{Dox}, 10 mg/kg, corresponding to 1 mg/kg of doxorubicin, -▪-), respectively; FIG. 8b is a graph showing the results of measuring weight of the tumor tissues excised at the end of the experiment of FIG. 8a (in the graph of FIGS. 8a and 8b, the values represent mean±SD, <0.05, **P<0.01, ***P<0.001 by ANOVA analysis); FIG. 8c is a series of photographs of the tumor tissues excised from each test group of FIG. 8b; FIG. 8d is a series of fluorescent microscopic images of tumor tissue sections obtained from tumor tissues of PC3-bearing cancer model mice, wherein the tumor tissues were excised 24 hour after intravenous injection of 3 mg/kg doxorubicin to the PC3-bearing mouse model, which were taken by confocal fluorescence microscope in order to analyze drug distribution in the tumor tissues; and FIG. 8e is a schematic diagram illustrating the effect of the recombinant exosome presenting PH20 on the surface and the combined treatment of the recombinant exosome with the embedded doxorubicin therein according to an embodiment of the present invention.

### DETAILED DESCRIPTION OF THE INVENTION

### Definitions

The term “exosome” as used herein is a nano-sized cell-derived vesicle that may be present in all biological fluids, including, perhaps, blood, urine, and cell culture medium. Exosomes are known to be between 30 and 100 nm in size, and they are known to be secreted from the cell or secreted directly through the cell membrane, when multivesicular bodies fuses with the cell membrane. Exosomes are known to play an important role in a variety of processes such as clotting, intercellular signaling, and metabolic waste management.

The term “recombinant exosome” as used herein refers to an artificially produced exosome and it is an exosome isolated from a culture of a transduced host cell prepared by transducing a host cell capable of producing exosomes with a gene encoding a heterologous protein by genetic engineering. The culture may be cell culture medium of the transduced host cell or the transduced host cell itself. The expressed heterologous protein may exist in the inner spacer or on the surface of the exosome.

The term “hyaluronidase” as used herein refers to an enzyme that catalyzes the degradation of hyaluronic acid (HA). In 1971, Karl Meyer classified this enzyme into three groups based on their enzymatic reaction products. The three major types are the two eukaryotic endo-glucosidase hydrolases and prokaryotic lyase-type glucosidases. There are five functional hyaluronidases: HYAL1, HYAL2, HYAL3, HYAL4 and HYAL5 and one pseudogene HYAL6. The HYAL5 is known as sperm adhesion molecule 1 (SPAM1) or PH20, The surface protein PH20 belongs to the glycosyl hydrolase family 56 and is expressed in the testes. PH20 randomly hydrolyzes (1→4) linkages between N-acetyl-β-D-glucosamine and D-glucuronate residues in the hyaluronic acid. SPAM-1/PH20 is involved in sperm-oocyte attachment. At fertilization, the sperm first must penetrate the cumulus cell layer surrounding the egg before it reaches the zona pellucida, however the egg is enclosed in a matrix containing the hyaluronic acid formulated before ovulation. PH20 supports the digestion of the hyaluronic acid so that sperm penetrates the layers of cumulus cells.

The term “immunogenic cell death” refers to a type of cell death caused by cell growth inhibitors such as anthracyclines, taxan-based chemotherapeutic agents, oxaliplatin and bortezomib, radiotherapy or photodynamic therapy. Unlike general apoptosis, the immunogenic cell death can induce an effective anticancer immune response through activation of dendritic cells and activation of specific T cell responses thereby. A substance inducing immunogenic cell death is called an immunogenic cell death inducer or an immunogenic cell death-inducing chemotherapeutic agent. The immunogenic cell death and the immunogenic cell death inducers are well described in a prior art (Kroemer et al., Annu. Rev. Immunol., 31: 51-72, 2013). This document is incorporated herein by reference in its entirety.

The term “non-immunogenic cell death-inducing anticancer agent” as used herein refers to a substance that induces general cell death, not immunogenic cell death.

The term “anthracycline-based anticancer agent” as used herein refers a type of anticancer chemotherapeutic agents independent on cell cycle, which are derived from Streptomyces peucetius var. caesius. Anthracycline-based anticancer agents are used for the treatment of various cancers including leukemia, lymphoma, breast cancer, stomach cancer, uterine cancer, ovarian cancer, bladder cancer and lung cancer. The first discovered anthracycline-based anticancer was daunorubicin, followed by doxorubicin, followed by epirubicin, idarubicin, pixantrone, Sabarubicin, valrubicin, and the like. Examples of the mechanism of action of the anthracycline-based anticancer agent include inhibiting DNA and RNA synthesis and inhibiting the proliferation of rapid growing cancer cells thereby by inserting between the base-pairing of the DNA/RNA strand, inhibiting the activity of the topoisomerase II enzyme, inhibiting transcription and replication and inhibiting the relaxation of supercoiled DNA thereby by inhibiting topoisomerase II activity, inducing damages of DNA, protein and cell membrane through the formation of iron-mediated free oxygen radicals, and inducing histone expulsion from chromatin which controls the epigenome and transcriptomes. Recent studies have shown that doxorubicin increases the Th1 immune response by activating CD4⁺ cells (Park et al., Int. Immunopharmacol. 9(13-14): 1530-1539, 2009), and it has been reported that combined administration of dendritic cells and doxorubicin induced immunogenic cell death of osteosarcoma (Kawano et al., Oncol. Lett. 11: 2169-2175, 2016).

The term “taxanoid anticancer agent” or “taxane anticancer drug” as used herein refers to diterpenoid taxane derivatives extracted from genus of Taxus. It is a mitotic inhibitor with a mechanism of promoting assembly and inhibiting disassembly. Paclitaxel is a taxane-based anticancer drug extracted from the periderm of Taxus brevifolia. In 1992, it was approved by the US FDA for the treatment of intractable ovarian cancer, and docetaxel is a taxane-based anticancer approved by the US FDA which was derived from Taxus bacaata and has similar efficacy to paclitaxel. It is used for the treatment of breast cancer, non-small cell lung cancer, lymphoma, bladder cancer and the like, and has high hydrophilic properties compared with paclitaxel. Recently, a taxane-based anticancer agent has been shown to have a mechanism of promoting immunogenic cell death of these cancer cells by sensitizing cancer cells to cytotoxic T lymphocytes.

The term “immune checkpoint inhibitor” as used herein refers to a class of drugs that block certain types of immune system cells, such as T lymphocytes, and certain proteins produced by some cancer cells, which prevent T lymphocytes from killing cancer cells. Thus, when these proteins are blocked, the “brake system” of the immune system is released and T lymphocytes can kill cancer cells better. PD-1/PD-L1 and CTLA-4/B7-1/B7-2 are well known as the above-mentioned “immune check point”. Examples of PD-1 inhibitors include Pembrolizumab (trademark: Keytruda), Nivolumab (trademark: Opdivo), and the inhibitors of PD-L1, a ligand of PD-1 includes Atezolizumab (trademark: Tecentrig) and Avelumab (trademark: Bavencio). Meanwhile, Ipilimumab (trademark: Yervoy) and the like have been approved by the US FDA as CTLA-4 inhibitors that inhibit the interaction of CTLA-4/B7-1/B7-2. Clinical trials in recent years have been an impressive success in the treatment of patients suffering from some cancers, particularly metastatic melanoma or Hodgkin lymphoma, and there is much potential for clinical trials in other types of cancer patients.

### DETAILED DESCRIPTION OF THE INVENTION

According to one aspect of the present invention, there is provided a recombinant exosome that presents a hyaluronidase on its surface.

In the recombinant exosome, the hyaluronidase may be a GPI-anchored membrane-bound hyaluronidase, and the GPI-anchored form membrane-bound hyaluronidase may be a full-length PH20 and the PH20 may include any one of the amino acid sequences of SEQ ID NOS: 1 to 30. The amino acid sequences shown in SEQ ID NOS: 1 to 30 may be recombinant human PH20 or a predicted protein derived from other species having a homology of at least 85% or more, which corresponds to 36-490 a.a. of human PH20, as shown in Table 1.

According to another aspect of the present invention, there is provided a recombinant exosome that presents a hyaluronidase on its surface and encapsulates an anticancer agent therein.

In the recombinant exosome, the anticancer compound may be an immunogenic cell death-inducing chemotherapeutic agent, an immune checkpoint inhibitor, a mitotic inhibitor, an antimetabolite, a hormone agent, an alkylating agent, Flt3 ligands or topoisomerase inhibitors.

In the recombinant exosome, the immunogenic cell death-inducing chemotherapeutic agent may be selected from the group consisting of an anthracycline-based anticancer agent, a taxane-based anticancer agent, an anti-EGFR antibody, a BK channel agonist, bortezomib, a cardiac glycoside, a GADD34/PP1 inhibitor, LV-tSMAC, Measles virus or oxaliplatin, and the cardiac glycoside may be used in combination with a non-immunogenic cell death-inducing chemotherapeutic agent and the GADD34/PP1 inhibitor may be used in combination with mitomycin. The anthracycline-based anticancer agent may be used in combination with mitomycin, and the anthracycline-based anticancer agent may be daunorubicin, doxorubicin, epirubicin, idarubicin, pixantrone. In addition, the taxanoid anticancer agent may be paclitaxel or docetaxel, and immunogenic cell death-inducing chemotherapeutic agent may be used in combination with the immune checkpoint inhibitors.

In the recombinant exosome, the immune checkpoint inhibitor may be a PD-1/PDLL interaction inhibitor or a CTLA-4/B7-1/B7-2 interaction inhibitor and the PD-1/PD-L1 interaction inhibitor may be Pembrolizumab, Nivolumab, Atezolizumab or Avelumab, and the CTLA-4/B7-1/B7-2 interaction inhibitor may be Ephilimumab (Ipilimumab).

In the recombinant exosome, the alkylating agent may be selected from the group consisting of cyclophosphamide, mechlorethamine, chlorambucil, melphalan, carmustine (BCNU), lomustine (CCNU), ifosfamide, procarbazine, dacarbazine (DTIC), temozolomide, altretamine, cisplatin, carboplatin and oxaliplatin.

In the recombinant exosome, the topoisomerase inhibitor may be topotecan or irinotecan (CPT II), and the metabolic antagonist may be deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate, asparagine, asparaginase or pemetrexed.

In the recombinant exosome, the mitotic inhibitor may be selected from the group consisting of vincristine, vinblastine, vinorelbine, paclitaxel, docetaxel, estramustine phosphate and NAB-paclitaxel.

In the recombinant exosome, the hormone agent may be selected from the group consisting of tamoxifen, aromatase inhibitor, leuprolide, bicalutamide, flutamide, nilutamide and octreotide.

According to another aspect of the present invention, there is provided a composition for treating cancer comprising the recombinant exosome as an active ingredient.

According to another aspect of the present invention, there is provided a composition for treating cancer comprising a recombinant exosome that presents a hyaluronidase on its surface and encapsulating an anticancer compound therein as an active ingredient.

According to another aspect of the present invention, there is provided a composition for treating cancer comprising a recombinant exosome that presents a hyaluronidase on its surface and an anticancer compound as an active ingredient.

In the composition for treating cancer, the anti-cancer compound may be an immunogenic cell death-inducing chemotherapeutic agent, an immune checkpoint inhibitor, a mitotic inhibitor, an antimetabolite, a hormone agent, an alkylating agent, Flt3 ligands or topoisomerase inhibitors.

In the composition for treating cancer, the immunogenic cell death-inducing chemotherapeutic agent may be selected from the group consisting of an anthracycline-based anticancer agent, a taxane-based anticancer agent, an anti-EGFR antibody, a BK channel agonist, bortezomib, a cardiac glycoside, a GADD34/PP1 inhibitor, LV-tSMAC, Measles virus or oxaliplatin, and the cardiac glycoside may be used in combination with a non-immunogenic cell death-inducing chemotherapeutic agent and the GADD34/PP1 inhibitor may be used in combination with mitomycin. The anthracycline-based anticancer agent may be used in combination with mitomycin, and the anthracycline-based anticancer agent may be daunorubicin, doxorubicin, epirubicin, idarubicin, pixantrone. In addition, the taxanoid anticancer agent may be paclitaxel or docetaxel, and immunogenic cell death-inducing chemotherapeutic agent may be used in combination with the immune checkpoint inhibitors.

In the composition for treating cancer, the immune checkpoint inhibitor may be a PD-1/PD-L1 interaction inhibitor or a CTLA-4/B7-1/B7-2 interaction inhibitor and the PD-1/PD-L1 interaction inhibitor may be Pembrolizumab, Nivolumab, Atezolizumab or Avelumab, and the CTLA-4/B7-1/B7-2 interaction inhibitor may be Ephilimumab (Ipilimumab).

In the composition for treating cancer, the alkylating agent may be selected from the group consisting of cyclophosphamide, mechlorethamine, chlorambucil, melphalan, carmustine (BCNU), lomustine (CCNU), ifosfamide, procarbazine, dacarbazine (DTIC), temozolomide, altretamine, cisplatin, carboplatin and oxaliplatin.

In the above composition for treating cancer, the topoisomerase inhibitor may be topotecan or irinotecan (CPT II), and the metabolic antagonist may be deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate, asparagine, asparaginase or pemetrexed.

In the composition for treating cancer, the mitotic inhibitor may be selected from the group consisting of vincristine, vinblastine, vinorelbine, paclitaxel, docetaxel, estramustine phosphate and NAB-paclitaxel.

In the composition for treating cancer, the hormone agent may be selected from the group consisting of tamoxifen, aromatase inhibitor, leuprolide, bicalutamide, flutamide, nilutamide and octreotide.

The composition of the present invention may contain a pharmaceutically acceptable carrier. The composition comprising a pharmaceutically acceptable carrier may be various oral or parenteral formulations, but is preferably a parenteral formulation. In the case of formulation, a diluent or excipient such as a commonly used filler, an extender, a binder, a wetting agent, a disintegrant, a surfactant or the like may be used. Solid form preparations for oral administration include tablets, pills, powders, granules, capsules and the like, which may contain at least one excipient such as starch, calcium carbonate, sucrose or lactose, gelatin, etc. In addition to simple excipients, lubricants such as magnesium stearate, talc, and the like may also be used. Liquid preparations for oral administration include suspensions, solutions, emulsions, and syrups. Various excipients such as wetting agents, sweetening agents, fragrances, preservatives, etc. may be included in addition to water and liquid paraffin. Formulations for parenteral administration include sterile aqueous solutions, non-aqueous solutions, suspensions, emulsions, freeze-dried preparations, and suppositories. Examples of the non-aqueous solution and the suspension may include propylene glycol, polyethylene glycol, vegetable oil such as olive oil, and injectable ester such as ethyl oleate. As a base for suppositories, witepsol, macrogol, Tween 61, cacao paper, laurin, and glycerogelatin may be used.

The compositions may be in one formulation selected from the group consisting of tablets, pills, powders, granules, capsules, suspensions, solutions, emulsions, syrups, sterile aqueous solutions, non-aqueous solutions, suspensions, emulsions, lyophilized preparations and suppositories.

The composition of the present invention may be administered orally or parenterally. When administered parenterally, it can be administered through various routes such as intravenous injection, intranasal inhalation, intramuscular injection, intraperitoneal administration, and percutaneous absorption.

The composition of the present invention may be administered in a therapeutically effective amount.

The term “therapeutically effective amount” as used herein refers to an amount sufficient to treat a disease at a reasonable benefit/risk ratio applicable to medical treatment, and the effective dose level will depend on the type of subject and severity of the symptom, age, sex, sensitivity to the drug, time of administration, route of administration, rate of excretion, duration of treatment, factors including co-administered drugs, and other factors well known in the medical field. The composition of the present invention may be administered at a dose of 0.1 mg/kg to 1 g/kg, more preferably at a dose of 1 mg/kg to 500 mg/kg. On the other hand, the dosage can be appropriately adjusted according to the age, sex, and condition of the patient.

The composition of the present invention may be administered as an individual therapeutic agent or in combination with other active substances, and in the letter case it may be administered sequentially or simultaneously with the other active substances. And it may be administered singly or multiply. It is important to take into account all of the above factors and to administer the amount in which the maximum effect can be obtained in a minimal amount without side effects, and it can be determined by those skilled in the art.

According to another aspect of the present invention, there is provided a method for treating a subject suffering from cancer, comprising administering a therapeutically effective amount of the recombinant exosome to the subject.

In the method, one or more anticancer compounds may be further administered to the subject. In this case, the anticancer compound may be administered simultaneously with the recombinant exosome or sequentially administered at intervals.

In the method, a photodynamic therapy or a radiation therapy may be applied to the subject in place of the administration of the anticancer compound or in parallel with the administration of the anticancer compound.

According to another aspect of the present invention, there is provided a method for treating cancer, comprising administering therapeutically effective amount of a recombinant exosome that presents recombinant hyaluronidase on its surface to a subject suffering from cancer.

In the method, the photodynamic therapy or the radiotherapy may be additionally applied to the subject.

In the method, the anticancer compound is as described above.

TABLE 1
Available mammalian PH20

| | | | Position | Homo- |
|---|---|---|---|---|
| | | | of | logy |
| | | | corre- | with |
| SEQ | | | sponding | SEQ |
| ID | | NCBI or | amino | ID |
| NOs | Species | GenBank No. | acids | NO1 |
| 1 | Homo sapiens | NP_001167515.1 | 36-490 | — |
| 2 | Homo sapiens | AAC60607.2 | 36-490 | 99% |
| 3 | Pan troglodytes | XP_003318802.2 | 36-490 | 99% |
| 4 | Pan paniscus | XP_003815501 | 36-490 | 99% |
| 5 | Gorilla gorilla | XP_018886147.1 | 36-490 | 98% |
| 6 | Pan troglodytes | BAK62198.1 | 36-490 | 98% |
| 7 | Gorilla gorilla | XP_004046192.1 | 36-490 | 98% |
| 8 | Pongo abelii | XP_003777169.1 | 36-490 | 97% |
| 9 | Pongo abelii | XP_002818442.1 | 36-490 | 97% |
| 10 | Hylobates lar | ACV60218.1 | 36-490 | 97% |
| 11 | Symphalangus syndactylus | AEH95769.1 | 36-490 | 97% |
| 12 | Nomascus leucogenys | XP_003261317.1 | 36-490 | 96% |
| 13 | Nomascus leucogenys | XP_003261319.1 | 36-490 | 96% |
| 14 | Nasalis larvatus | AEX20364.1 | 36-491 | 93% |
| 15 | Colobus angolensis | XP_01187134.1 | 36-491 | 93% |
| 16 | Colobus guereza | AEX20365.1 | 36-491 | 93% |
| 17 | Semnopithecus entellus | ACV60223.1 | 36-491 | 92% |
| 18 | Colobus angolensis | XP_011807133.1 | 36-491 | 93% |
| 19 | Pygathrix nemaeus | ACV60224.1 | 36-491 | 92% |
| 20 | Rhinopithecus roxellana | XP_010366292.1 | 36-491 | 92% |
| 21 | Saguinus oedipus | AEX20362.1 | 36-491 | 89% |
| 22 | Cercopithecus mitis | ACV60221.1 | 36-491 | 89% |
| 23 | Macaca nemestrina | XP_011728213.1 | 36-491 | 89% |
| 24 | Aotus nancymaae | XP_012329390.1 | 36-491 | 88% |
| 25 | Saguinus fuscicollis | ACV60227.1 | 36-491 | 89% |
| 26 | Callimico goeldii | ACV60225.1 | 36-491 | 89% |
| 27 | Erythrocebus patas | ACV60222.1 | 36-491 | 90% |
| 28 | Chlorocebus aethiops | AEX20366.1 | 36-491 | 90% |
| 29 | Papio hamadryas | ACV60219.1 | 36-491 | 89% |
| 30 | Chlorocebus sabaeus | XP_007980982.1 | 36-491 | 89% |

Given the limitations of synthetic nano-formulations with truncated recombinant hyaluronidase, the present inventors have developed a recombinant exosome with native GPI-anchored PH20, which is a natural-derived nanoparticle in essence. Exosomes are nano-sized extracellular vesicles released by most cell types, including small RNAs, lipids and proteins, and may have several advantages over currently available synthetic drug delivery systems. These advantages include the ability to overcome natural barriers, unique cell targeting properties, and enhanced permeability and retention (EPR) effects and biocompatibility. On the basis of the original function of delivering biological information, the use of exosomes as therapeutic agents has received attention. It is known that GPI-anchored proteins are abundant in lipid rafts on the surface of exosomes during their in vivo formation and as a result it is relatively simple to present these proteins on the surface of exosomes. Therefore, it is expected that naturally occurring exosomes will act as enzyme nanoparticles themselves without any chemical modification.

The inventors have designed a new exosome-based platform that can penetrate tumor tissues by itself and perform drug delivery. Compared with previous studies using recombinant PH20, the inventors first demonstrated the high activity of GPI-anchored hyaluronidase on the surface of exosomes for the treatment of cancer. Thus, the present invention provides strong evidences and bases for the need for exosome that presents membrane-bound proteins on its surface. Enzyme-based drug delivery systems with native GPI immobilized proteins can be applied to both medical and research applications.

### BEST MODE FOR THE INVENTION

Hereinafter, the present invention will be described in more detail by following examples. It will be apparent to those skilled in the art that the present invention is not limited to the disclosed examples, but may be embodied in many different forms and the examples are provided in order to complete the disclosure of the present invention and to fully inform a skilled in the art.

### Experimental Method

The general laboratory method used in the present invention is as follows:

Cell Culture:

HEK293T cells were grown in Dulbecco's modified Eagle's medium (DMEM) supplemented with 10% fetal bovine serum and 1% antibiotic. Human PC3 prostate cancer cells and murine 4T1 breast cancer cells were maintained in RPMI medium supplemented with 10% fetal bovine serum and 1% antibiotic. All cell lines were maintained at 5% CO₂ at 37° C.

Immunoblotting:

The total amount of protein was determined by the BCA assay kit and the same amount (10 μg) of exosome protein was used for western blot analysis. Proteins were separated by SDS-PAGE and transferred to a nitrocellulose membrane. The membranes were blocked with 1×TBST (Tris-buffered saline, 0.05% tween 20) for 1 hour with 5% non-fat dry milk. The blot was incubated overnight at 4° C. with a primary antibody (anti-PH20 antibody, 1: 500, Abcam, ab196596; anti-Alix antibody, 1: 1000, Santacruz, sc99010; or anti-Tsg101 antibody, 1: 500, Santacruz, sc22774). The membrane was then reacted with HRP-conjugated anti-mouse or anti-rabbit secondary antibody (Sigma-Aldrich, USA) and the results visualized by chemiluminescence (Bio-Rad, USA). For quantitative analysis of PH20 in exosomes, recombinant PH20 was added as a control and the band volume was analyzed using ImageJ software.

Dynamic Light Scattering Analysis (DLS):

The size distribution of exosomes prepared using Zetasizer nano Zs (Malvern Instruments, Malvern, Worcestershire, UK) at a fixed angle of 25° to 173° was analyzed. Data were analyzed using software provided by the equipment.

Transmission Electron Microscopy (TEM):

To confirm the size and shape of the exosomes, 10 μg of exosomes loaded with 4% formaldehyde were loaded onto Formvar-carbon coated grids. After washing, the samples were stained with 1% (w/v) uranyl acetate for 1 minute. The lattice was dried and analyzed by transmission electron microscopy (Tecnai TEM).

Lipid Raft Domain Separation:

The lipid rafts fraction and non-raft fractions were separated from the exosomes by a slight modification of the protocol of the manufacturer (BioDynamics Laboratory Inc.). Briefly, exosomes were washed with cold PBS and incubated at room temperature with weak RIPA buffer (containing A-buffer, 1% NP-40) containing a protease inhibitor cocktail at 4° C. for 20 minutes. After centrifugation at 14,000 rpm for 20 minutes, the supernatant (RIPA-soluble fraction) was collected and the pellet was mixed with solubilization buffer (B-buffer) for 5 minutes at room temperature. The dissolved pallet was centrifuged at 14,000 rpm for 5 minutes and the supernatant (RIPA insoluble fraction) was collected. RIPA soluble fraction refers to cytoplasmic or non-raft membrane protein and RIPA insoluble fraction refers to membrane protein of lipid raft.

Enzyme Activity Assay:

The protocol of the manufacturer (Sigma, USA) was used to compare the enzyme activity of Exo-PH20 with the enzyme activity of recombinant human PH20 (rHuPH20). Specifically, after the diluted enzyme sample was stabilized at 37° C., the sample was immediately mixed with the hyaluronic acid and reacted at 37° C. for 45 minutes. The reaction was transferred to a cuvette containing the acidic albumin solution and allowed to react at room temperature for 10 minutes. Then, the transmittance of the sample was measured at 600 nm (UV/vis spectrophotometer, Beckman). Enzyme activity was calculated according to the protocol provided.

Particle Exclusion Assay:

To visualize HA expression of PC3 cell line in vitro, cells were incubated in a 4-well chamber or a 35 mm confocal dish for 24 hours and then incubated with serum-free culture medium containing Exo-PH20. Subsequently, the culture medium was replaced with a suspension of fixed mouse erythrocytes at 1×10⁹ cells/mL in PBS. Cells were imaged with a phase contrast microscope coupled with a camera scanner and imaging software (Diagnostic Instruments, Inc.). The length (an average of 5 points per cell) was determined by the distance between the cells and fixed erythrocytes. Values were calculated as the mean±SD of relative lengths to the untreated PC3 HA region (control, expressed as 100%).

In a real-time experiment, the PC3 cells were incubated with immobilized mouse erythrocytes and replaced with serum-free medium containing Exo-PH20. Images were obtained by real-time cell imaging microscopy (Biostation-IM, Nikon) every 5 minutes for 1 hour.

Evaluation of Anti-Cancer Effects in Mouse Model:

Male BALB/c nu/nu mice (6 weeks old) and female BALB/c mice (7 weeks old) were used and maintained at the KIST facility. This study protocol was approved by the KIST Institutional Animal Care and Use Committee (IACUC).

BALB/c nu/nu mice were inoculated with 1×10⁷ human PC3 prostate cancer cells in the left hind limb. 2×10⁶ murine 4T1 breast cancer cells were orthotopically inoculated into breast fat pads of female BALB/c mice. Tumor volume (mm³) was calculated as (width)²×(length)×0.5. After the tumors with an average size of 75 mm³ were stabilized, the experimental drugs were injected into each subject 5 times every 3 days into the tumor or intravenously and the tumor size was measured every 3 days. At the end of the experiment, the tumor was excised and weighed.

To analyze the enzymatic activity of exosomes in vivo, PBS, Exo-Con (5 mg/kg) and Exo-PH20 (5 mg/kg) were administered via intratumoral injection to PC3-bearing mice. After 6, 12, 24, 48 and 96 hours, mice were sacrificed and tumor tissues were excised and analyzed.

Distribution of Exosomes in Mice:

Exosomes were labeled with cy5.5 fluorescent dye to confirm the distribution of exosomes in the mice. Male BALB/c nu/nu mice were inoculated with 1×10⁷ human PC3 prostate cancer cells in the left hind limb. Mice with an average tumor size of 150 mm³ were injected intratumorally with PBS, 10 mg/kg cy5.5-Exo-Con and 10 mg/kg cy5.5-Exo-PH20, respectively. Whole body fluorescence images were obtained at 0, 1, 3, 6, 12, and 24 hours after the injection. At 24 hours, mice were sacrificed and the tumor tissues and organs (kidney, spleen, liver, heart, and Lung) were excised and the degree of accumulation of cy5.5-exosome was obtained through fluorescence imaging.

Distribution of Liposome-cy5.5:

Male BALB/c nu/nu mice were inoculated with 1×10⁷ human PC3 prostate cancer cells in the left hind limb. In tumors with an average size of 150 mm³, mice were intravenously injected with PBS, 10 mg/kg Exo-Con and 10 mg/kg Exo-PH20, respectively. After 3 hours, 4 mg/kg of liposome-cy5.5 was intravenously injected. After 7 hours, the mice were sacrificed and tumor tissues were excised to analyze the distribution of liposome-cy5.5.

Immunohistochemical Staining:

Tumor tissues were excised, fixed with 10% neutral formaldehyde overnight and embedded in paraffin. Paraffin-embedded tissues were sectioned and the sections were reacted overnight at 4° C. with anti-PH20 antibody (1: 200, Abcam, ab196596) or anti-HABP antibody (1:200, Abcam, ab181837). The next day, the sections were incubated with secondary antibodies (1:200, GBI Labs, D43-18) at room temperature for 2 hours and counterstained for 30 seconds. Images were obtained using an optical microscope (BX 51, Olympus, USA).

Immunofluorescent Staining:

Excised tumor tissues were frozen after embedded in OCT compound and sectioned. The sections were incubated overnight at 4° C. with CD8 antibody (1: 200, BD Pharmingen, bd550181) or anti-CD31 antibody (1:200, BD Pharmingen, bd553370) after blocking the sections (10 μm) with 3% BSA/PBS for 1 hour. The reacted sections were washed three times at an interval of 10 min and then incubated with Alexa-488-conjugated secondary antibody (1: 400, Jackson Immuno Research) for 1 hour at room temperature. Nuclei were stained with DAPI-Fluoromount-G. To analyze fluorescence distribution by doxorubicin in tumor tissue, cryo-sections of tumor tissues were incubated overnight with anti-CD31 antibody (1:200, BD Pharmingen, bd553370) at 4° C. After washing, they were incubated with Alexa-488-conjugated secondary antibody (1:400, Jackson Immuno Research) at room temperature for 1 hour. Nuclei were stained with DAPI-Fluoromount-G. Fluorescence signals of sections were obtained separately for each experimental group using a Leica fluorescence microscope.

Imaging of Doxorubicin Fluorescence Distribution in Tumor:

1×10⁷ human PC3 prostate cancer cells were inoculated into the left hind leg of male BALB/c nu/nu mice. Mice with an average tumor volume of 150 mm³ were injected intravenously with PBS, Exo-Con^{Dox} and Exo-PH20^{Dox}, respectively. After 24 hours, the mice were sacrificed and the tumor tissues were excised and analyzed.

Tumor Blood Flow:

Mice bearing human PC3 prostate cancer cells were used to detect changes in tumor blood flow. After this initial measurement (t=0), the experimental animals were intravenously injected with PBS, Exo-Con (10 mg/kg) and Exo-PH20 (10 mg/kg), respectively. Images were obtained at appropriate time points (t=1, 3, 6 and 24 hours) and changes in tumor blood flow were measured using a high resolution ultrasound imaging device (Vevo 770, Visual Sonics, Inc.).

### Example 1: Preparation of Exosome

HEK293T cells were inoculated at a density of 6×10⁶ cells per 150 mm dish. After replacing the medium with fresh serum-free DMEM medium supplemented with glutamax (final concentration 1%, Gibco), HEK293T cells were transfected with a plasmid DNA (PCMV6-HuPH20) containing the gene encoding the full-length PH20 (36-490 a.a., SEQ ID NO: 1) using lipofectamine 3000 (Invitrogen, USA). The supernatant was harvested and centrifuged with different RCFs to remove debris and microvesicles. Briefly, the supernatant was first centrifuged at 300×g for 10 min, followed by centrifugation at 2,000×g for 10 min and finally at 10,000 Xg for 30 min. Finally, after filtration through a 0.22 μm filter, the exosome containing supernatant was ultracentrifuged using 45 Ti rotor (Beckman Instruments) at 150,000 Xg for 3 hours. Exosomes were resuspended in PBS containing protease inhibitor (Roche) and stored at 4° C. Exosomes were obtained from cells not transfected with the PH20 gene and used as a control.

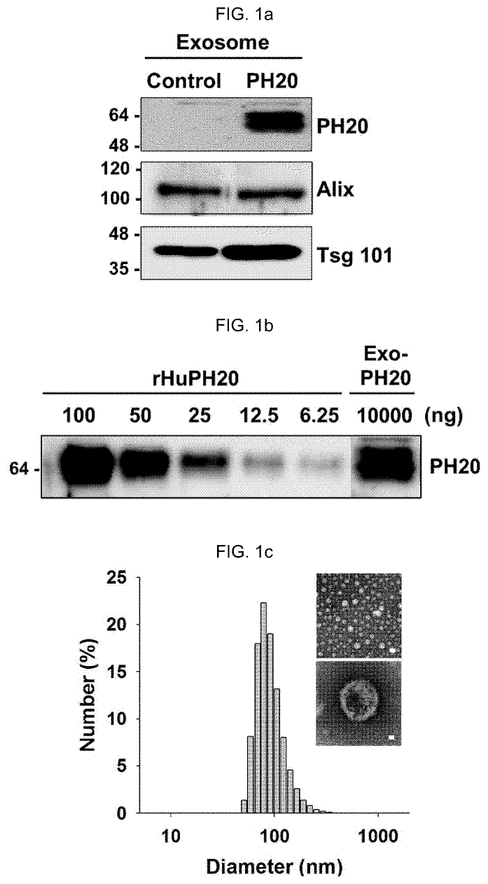

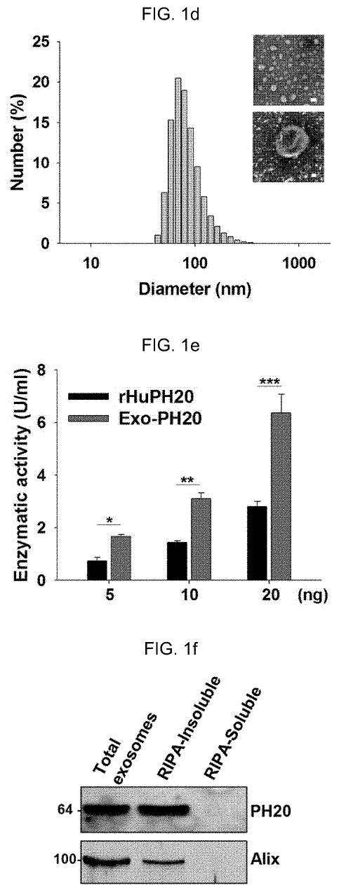

The purified Exo-PH20 contains an exosomal marker protein (Alix and Tsg101) and also PH20 on the membrane surface (FIGS. 1a and 1b). Transmission electron microscopy (TEM) images and dynamic light scattering (DLS) analysis of Exo-PH20 and Exo-con showed that both exosomes had complete sphere shape with an average size of 100 nm, and there was no difference between the two exosomes (FIGS. 1c and 1d).

Using a sequentially diluted concentration of recombinant human PH20 (rHuPH20) as a standard for quantitative western blotting, the concentration of PH20 protein in the exosomes was calculated using a calibration curve and found to be 5.3 μg per mg of exosomes (FIG. 1b). The PH20 hyaluronidase activity was quantitated by turbidity analysis and was found to be 1,954 U per mg of exosome (>360,000 U/mg PH20 in exosomes). This is a 3-fold increase compared to the control (rHuPH20 110,000 U/mg) (FIG. 1e and Table 2). In addition, as a result of the above-described lipid raft domain separation, it was confirmed that PH20 hyaluronidase was present in the lipid raft on the exosome (FIG. if). These results indicate that GPI-immobilized PH20 present in the lipid raft domain of exosomes has higher enzyme activity than the truncated form. Given that lateral migration of the GPI-anchored protein present in the lipid raft of exosomal membranes increases the activity of the protein, the microdomain-enriched exosomes are able to bind membrane-bound proteins and may be a suitable platform to present the membrane-bound proteins.

TABLE 2
Comparison of enzymatic activity of
recombinant human PH20 and PH20
presented on the surface of exosomes

| | PH20 | |
|---|---|---|
| | activity | |
| | (U/mg) | References |
| rHuPH20 | 110,000 | Thompson et al. Mol. Cancer Ther. 9 (11): |
| | | 3052-3064, 2010 |
| | | Zhou et al., Nano Lett., 9 (9): 3268-3274, 2009 |
| Exo-PH20 | 361,852 | The present invention |

### Example 2: Preparation of Doxorubicin-Loaded Exosomes

The present inventors hypothesized that anti-tumor efficacy would be enhanced if a chemotherapeutic drug such as doxorubicin (Dox), which is widely used in solid tumor treatment, is delivered simultaneously with PH20. Doxorubicin was encapsulated in exosomes by simple incubation with a final loading of 10.2±0.3%.

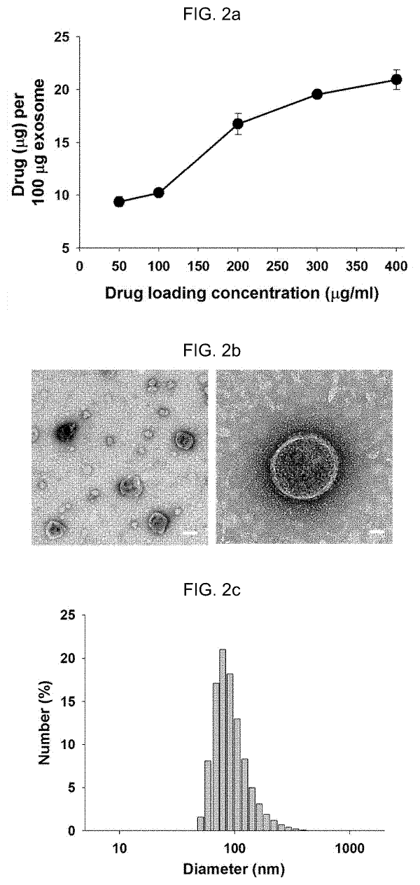

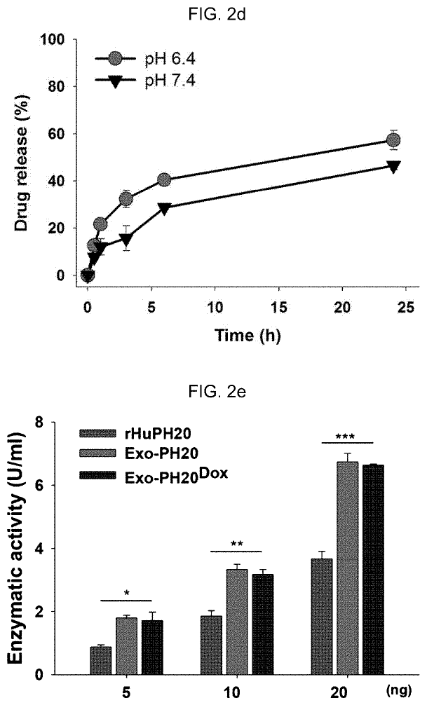

100 μg of exosomes prepared in the above Example 1 were mixed with doxorubicin hydrochloride (50, 100, 200, 300 and 400 μg) overnight at 4° C. to encapsulate doxorubicin (Dox) in exosomes. The unloaded drug was removed by air-fuge and the drug-loaded exosomes were diluted with PBS buffer. The loading of the drug was determined by measuring the fluorescence intensity of Dox (excitation at 480 nm and emission at 590 nm). The drug was added to the exosomes, gently mixed and immersed in PBS (pH 7.4) or acetic acid solution (pH 6.4) at 37° C. At predetermined time points (30 minutes, 1, 2, 3, 6 and 24 hours), the buffer was withdrawn for fluorescence analysis and replaced with fresh buffer. The amount of Dox released was determined by fluorescence analysis with excitation at 480 nm and emission at 590 nm. As a result, it was confirmed that the concentration of mixed doxorubicin and the amount of drug (μg) loaded per 100 μg of exosomes increased depending on the concentration of mixed doxorubicin (FIG. 2a).

The TEM and DLS analyzes of Exo-PH20 (Exo-PH20^{Dox}) containing doxorubicin showed no significant difference in size and shape compared to Exo-PH20, confirming that the drug loading did not alter the physical properties of the exosome (FIGS. 2b and 2c). The present inventors also examined the release of doxorubicin from Exo-PH20^{Dox} at acidic pH (pH 6.4), which is similar in the tumor microenvironment, rather than the physiological condition (pH 7.4), confirming that the release of doxorubicin was accelerated under acidic conditions (FIG. 2d). The PH20 hyaluronidase activity of Exo-PH20^{Dox} was similar to the PH20 hyaluronidase activity of Exo-PH20, confirming that the drug loading did not alter PH20 hyaluronidase activity (FIG. 2e).

### Experimental Example 1: Anticancer Activity of PH20 Surface Expression Exosomes

1-1: In Vitro Analysis

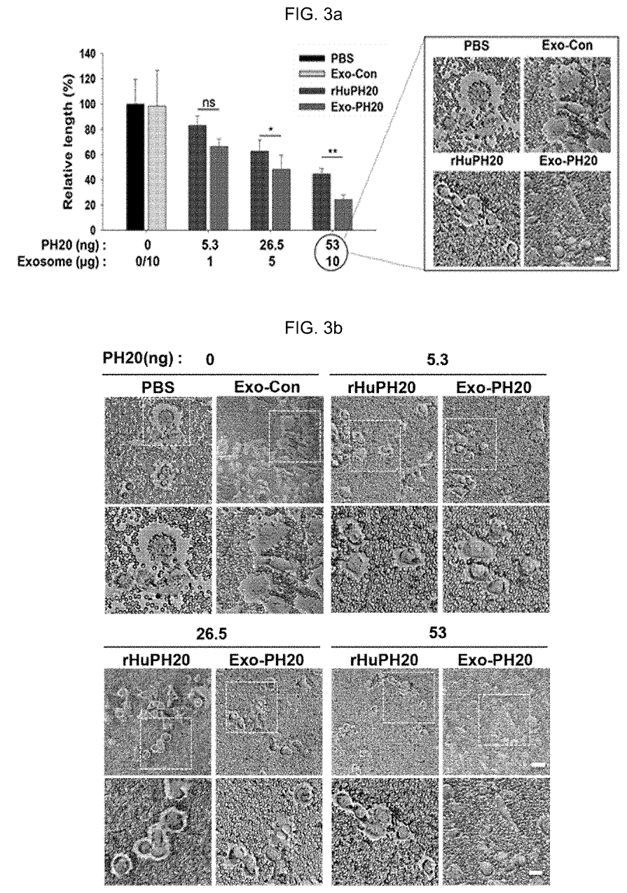

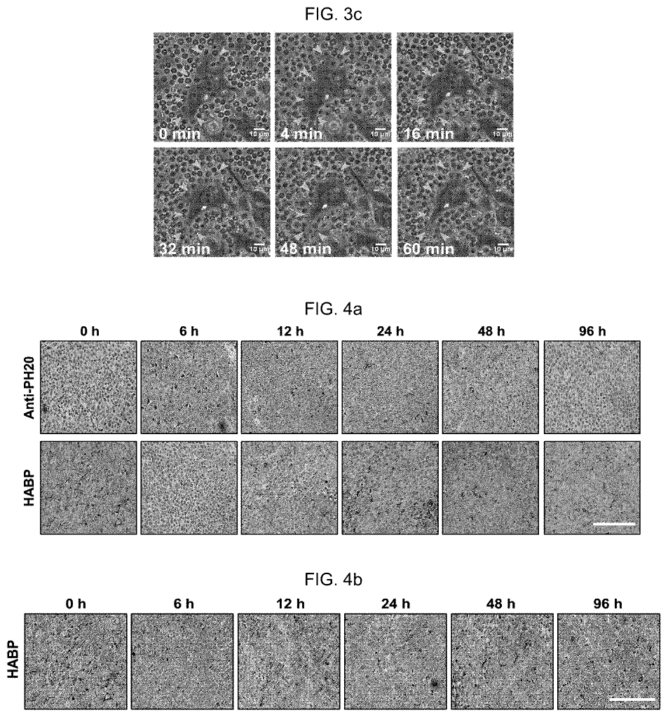

To confirm that the PH20-surface presenting exosomes have a high enzyme activity relative to human recombinant PH20, the present inventors compared degradation ability of rHuPH20 and Exo-PH20 converted to the same mass of the rHuPH20 on the extracellular matrix of PC3 human prostate cancer cell capable of producing a hyaluronic acid (HA)-dependent extracellular matrix. Specifically, in order to visualize the HA extracellular matrix in vitro, particle exclusion analysis was carried out using fixed red blood cells as described above. The HA-high extracellular matrix of PC3 cells was depleted in a concentration-dependent manner after treatment with recombinant human PH20 and Exo-PH20, but Exo-PH20 depleted the extracellular matrix more than recombinant human PH20. However, Exo-Con did not show such an effect (FIGS. 3a and 3b). Time-course experiments with PC3 cells showed that HA was substantially reduced within 60 min after Exo-PH20 treatment (FIG. 3c).

1-2: Intratumoral In Vivo Analysis

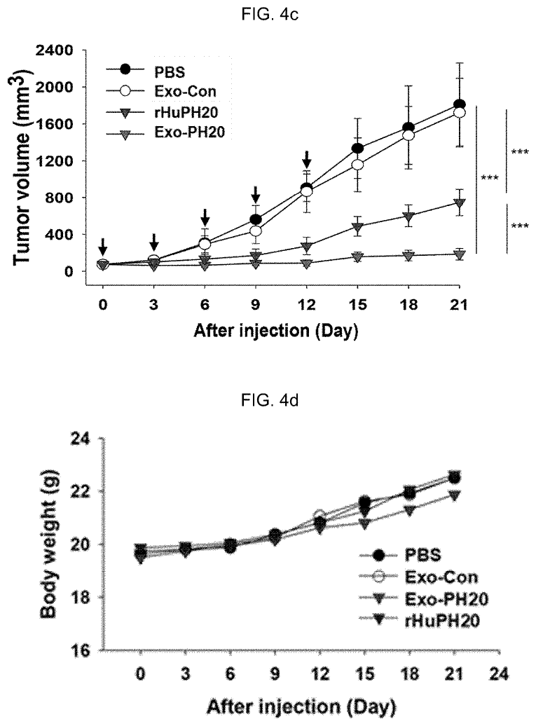

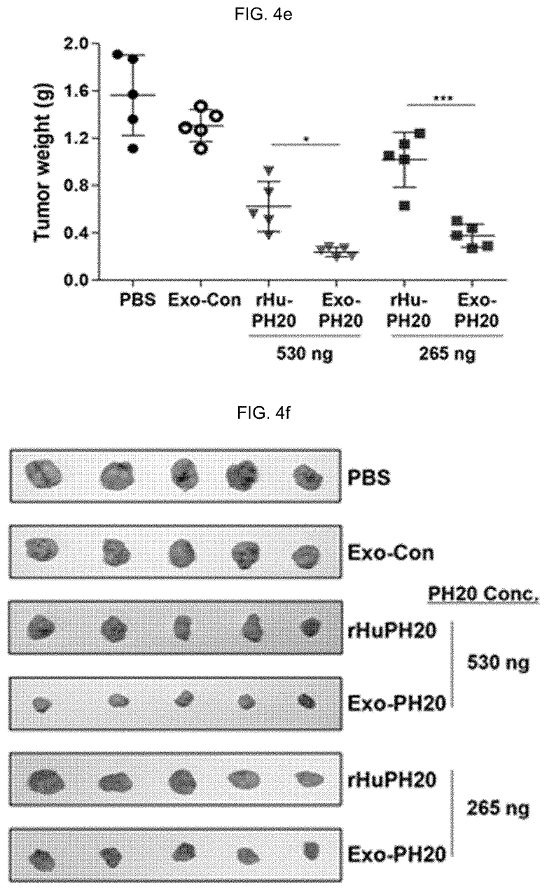

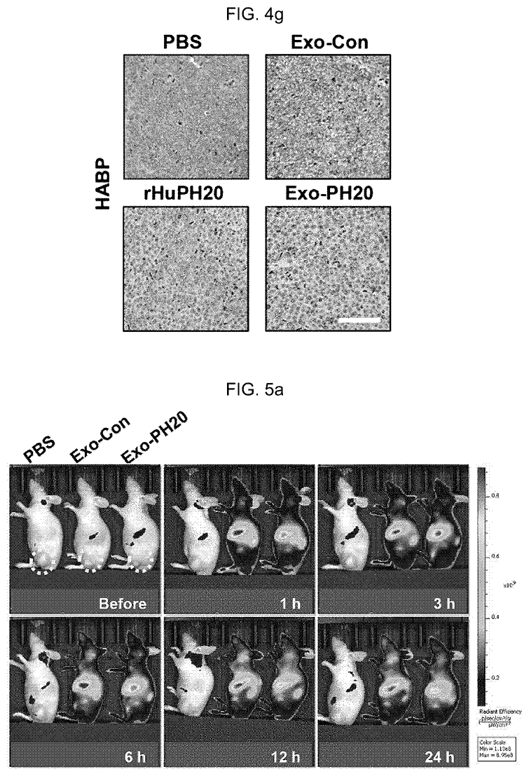

The anticancer activity of Exo-PH20 depleting HA was also assessed in PC3 (HA^{high}) cell-bearing heterologous xenograft model mice. A single intratumoral administration of Exo-PH20 (PH20 protein, 26.5 μg/kg; exosome, 5 mg/kg) effectively removed HA from the tumor ECM within 6 h post-administration (FIGS. 4a and 4b). This Exo-PH20-mediated HA depletion was sustained for >48 h with gradual reconstitution of HA.

On the other hand, since this Exo-PH20-mediated ECM remodeling could potentially inhibit tumor growth in vivo, the present inventors evaluated the antitumor activity of Exo-PH20 using PC3 xenograft mouse models. PBS, Exo-Con, rHuPH20 (530 ng) and Exo-PH20 (in an amount corresponding to 530 ng of rHuPH20) were injected intratumorally into mice every three days for a total of five injections, respectively, in order to counteract HA resynthesis and turnover. Exo-PH20 treatment slowed tumor growth compared to human recombinant PH20 converted to the same mass, whereas no substantial inhibition was observed in the control group (Exo-Con) and treatment of exosome and human recombinant PH20 did not affect the weight of the mice (FIGS. 4c and 4d). The mean weight of the excised tumor tissues was significantly lower in the Exo-PH20-treated group than in the control group, and the size of the excised tumor tissues was also smaller in the Exo-PH20-treated group than in the control group (FIGS. 4e and 4f). The HABP staining results showed that the HA levels of the PC3 xenograft mice were depleted in the tumors excised from the Exo-PH20 treated mice (FIG. 4g). Similar to the results of the extracellular matrix degradation (FIG. 3a), Exo-PH20 showed a superior tumor growth inhibitory effect compared with the recombinant human PH20, which was converted to the same mass. It suggests that Exo-PH20 can reduce tumor growth or expansion by affecting the ECM of tumor when HA was depleted in HA-expressing tumor-bearing mice.

1-3: Intravenous Administration In Vivo Analysis

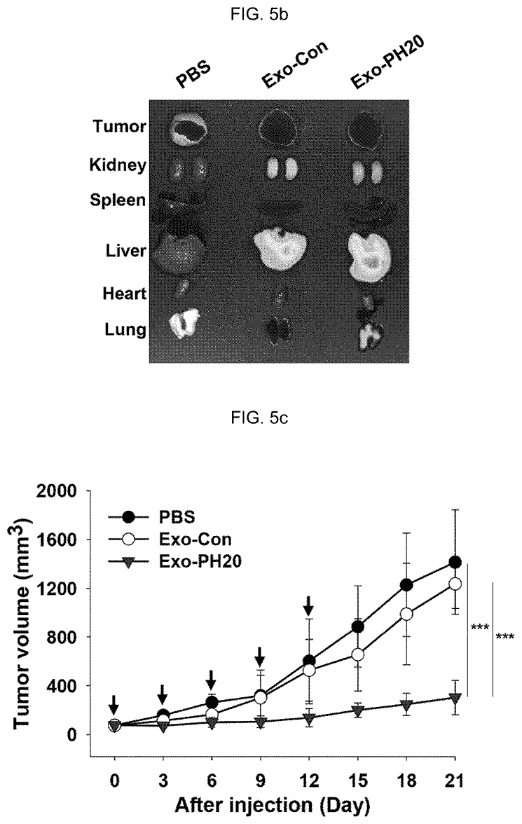

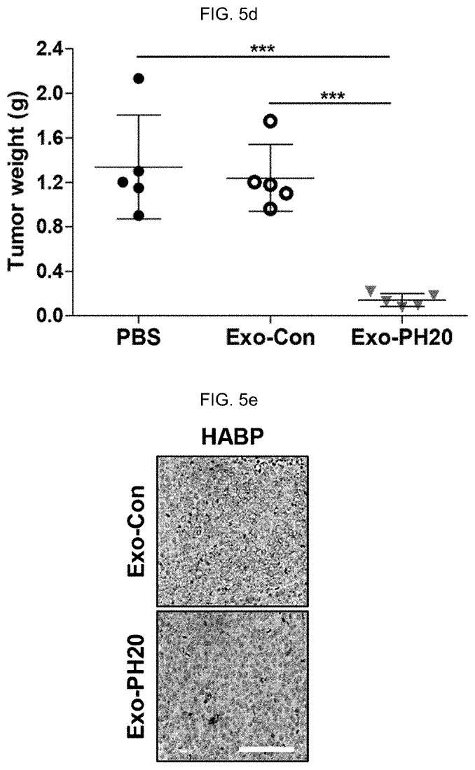

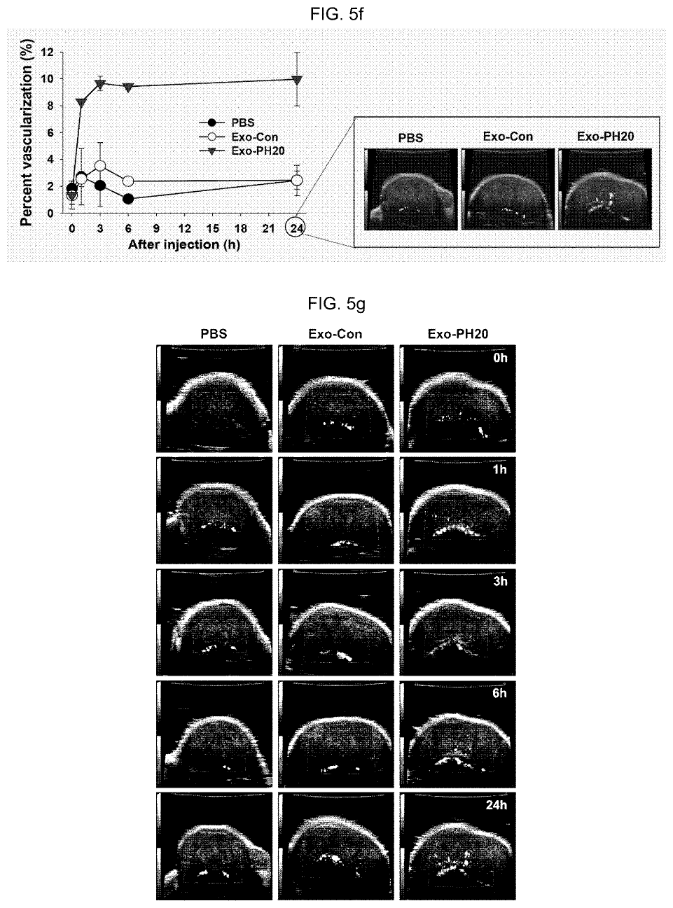

Next, the present inventors examined the distribution of Exo-PH20 administered intravenously in PC3 tumor-bearing mice prior to investigating anti-tumor efficacy of intravenously administered Exo-PH20. Although exosomes labeled with cy5.5-fluorescent dyes accumulate in the liver over time, it was found that they migrated to cancer by the EPR effect and they were accumulated in the tumor tissues and organs excised 24 hours after intravenous administration (FIGS. 5a and 5b).

Based on these results, the present inventors examined the anti-tumor efficacy of Exo-PH20 administered intravenously in PC3 tumor-bearing mice. The results showed that mice in the group treated with Exo-PH20 showed significantly reduced tumor growth (about 83% inhibition) compared to the control group (FIGS. 5c and 5d). The HABP staining results showed that HA levels of the PC3 xenografts mice were depleted in tumors excised from Exo-PH20 treated mice (FIG. 5e). The present inventors also investigated whether the decrease in HA in tumor tissues correlates with changes in tumor vascular reperfusion in vivo using high resolution ultrasound imaging. As shown in FIG. 5f, Exo-PH20 treatment resulted in a 3-fold increase in relative vascularization after 3 hours of exosome administration to PC3 tumor-bearing mice (FIG. 5g).

1-4: Nanoparticle Penetration Analysis

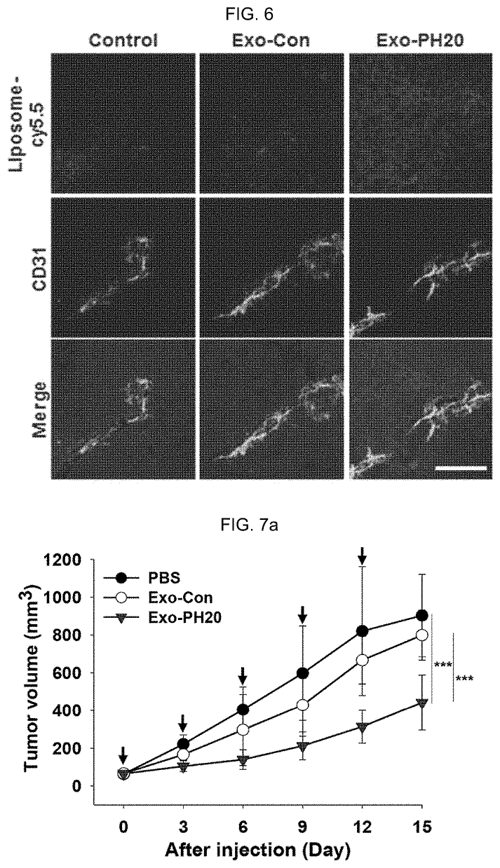

Based on these results, the present inventors further investigated whether Exo-PH20-mediated tumor ECM reconstruction could enhance nanoparticle penetration in HA-depleted tumor tissues. To evaluate the penetration of nanoparticles after HA depletion, fluorophore-conjugated PEG-liposomes were intravenously injected into PC3 tumor-bearing mice in the absence or presence of Exo-PH20 pretreatment. Liposome accumulation was minimal in untreated tumor tissue, but increased dramatically after Exo-PH20 treatment (FIG. 6). These results demonstrate that Exo-PH20 not only increased blood perfusion but also decreased interstitial fluid pressure in the tumor microenvironment due to HA depletion, thereby improving nanoparticle penetration in tumor tissues.

1-5: Analysis Using Animal Models of Immune Ability

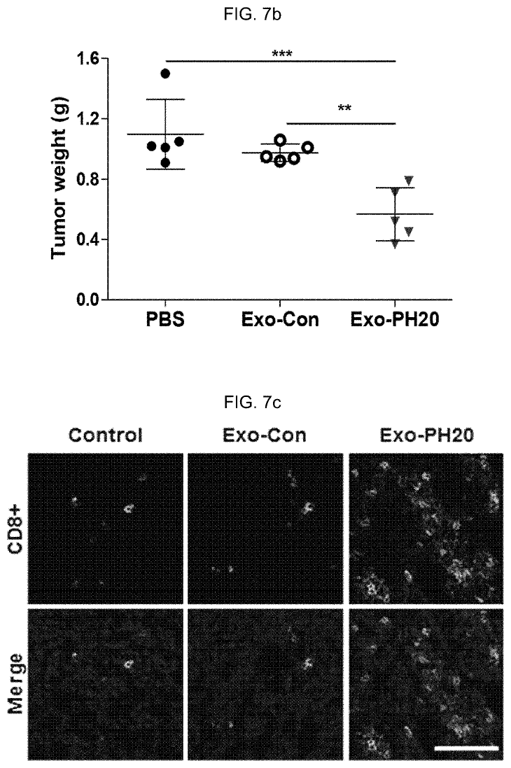

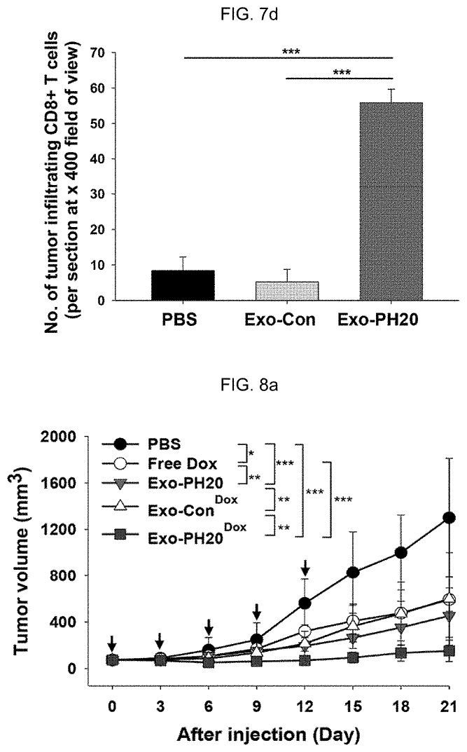

The present inventors then examined the efficacy of exo-PH20 administered systemically in immunocompetent mice. Compared with the control, tumor growth was significantly reduced by Exo-PH20 administration in BALB/c immunocompetent mice inoculated with 4T1 cells (FIGS. 7a and 7b). The present inventors then performed immunofluorescence staining of tumor tissues in order to analyze the presence of localized tumor invading CD8⁺ T cells. In the 4T1 tumor-bearing mouse model, extensive T cell infiltration was observed in tumor tissues upon administration of Exo-PH20 as compared to the control (FIGS. 7c and 7d). Taken together, these results indicate that Exo-PH20 successfully degrades HA in tumor microenvironment and enhances penetration of nanoparticles and immune cells in the tumor tissues.

### Experimental Example 2: Anticancer Activity of Recombinant Exosomes Presenting PH20 on the Surface and Encapsulating Doxorubicin

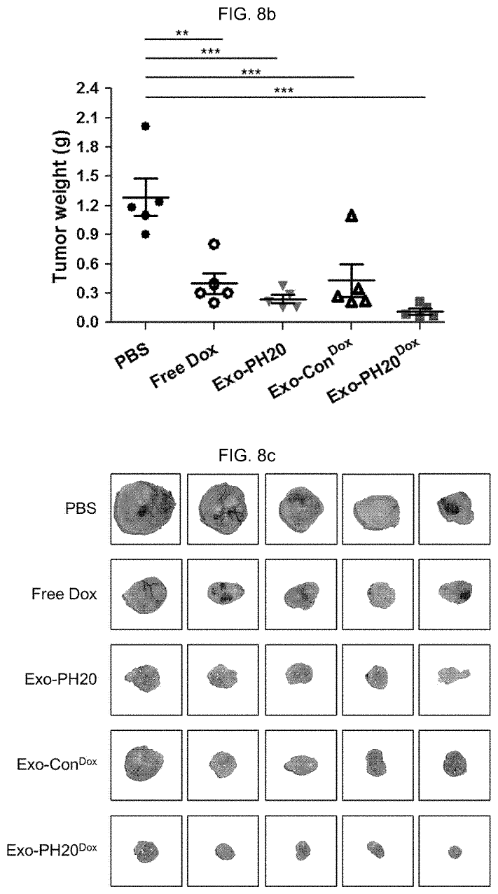

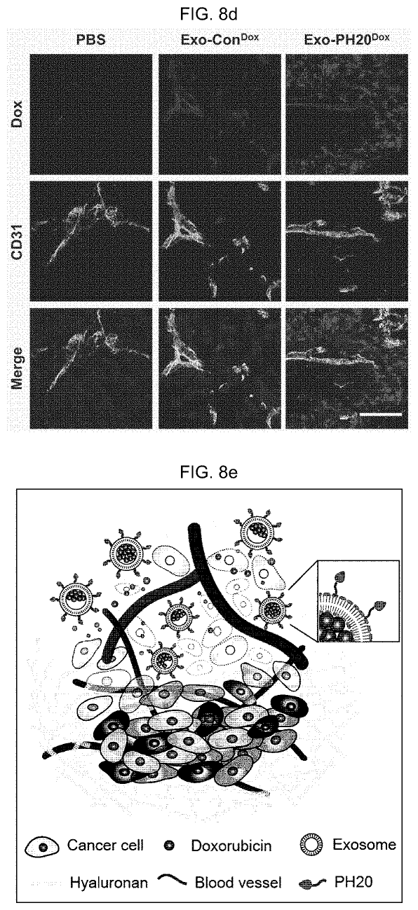

The possibility of treating Exo-PH20^{Dox} prepared in the above Example 2 was analyzed in vivo using PC3 xenografts. Test results show that treatment with Exo-PH20^{Dox} synergistically inhibited tumor growth, which means that combination therapy significantly improves anticancer efficacy (FIGS. 8a-8c). In particular, enhanced penetration of Exo-PH20 ensured effective drug release in the tumor tissues. A significantly higher and uniformly distributed Dox fluorescence signal was observed in blood vessels in the tumor tissue sections of tumor-bearing mice administered with Exo-PH20^{Dox} (FIG. 8d). Taken together, Exo-PH20 can enhance the efficacy of chemotherapy, which may be the result of depletion of HA by PH20, ECM reconstruction of subsequent tumors, and intratumoral infiltration of doxorubicin through increased vascular perfusion (FIG. 8e).

Exosomes presenting PH20 on its surface according to one embodiment of the present invention can be used to degrade the hyaluronic acid network of ECM, a component of the microenvironment surrounding cancer, and to improve the accessibility of cancer cells and anticancer compounds to cancerous tissues.

While the present invention has been described with reference to exemplary embodiments, it is to be understood that the invention is not limited to the disclosed exemplary examples, and on skilled in the art may comprehend that there are various modifications and equivalent examples. Accordingly, the true scope of the present invention should be determined by the technical idea of the appended claims.

What is claimed is:

1. A recombinant exosome that presents a hyaluronidase on its surface.

2. The recombinant exosome of claim 1, wherein the hyaluronidase is a GPI-anchored form of membrane-bound hyaluronidase.

3. The recombinant exosome of claim 2, wherein the GPI-anchored form of the membrane-bound hyaluronidase is a full-length PH20.

4. The recombinant exosome of claim 3, wherein the PH20 comprises any one of the amino acid sequences set forth in SEQ ID NOs: 1-30.

5. A recombinant exosome that presents a hyaluronidase on its surface and encapsulate an anticancer agent therein.

6. The recombinant exosome of claim 5, wherein the anticancer compound is an immunogenic cell death agent, an immune checkpoint inhibitor, a mitotic inhibitor, an antimetabolite, a hormone, an alkylating agent, a Flt3 ligand or a topoisomerase inhibitor.

7. The recombinant exosome of claim 5, wherein the immunogenic cell death-inducing chemotherapeutic agent is selected from the group consisting of anthracycline-based anticancer agents, cetuximab, taxane-based anticancer agents, bleomycin, cyclophosphamide, cardiac glycosides, GADD34/PP1 inhibitors, mitoxantrone and oxaliplatin.

8. The recombinant exosome of claim 7, wherein the anthracycline-based anticancer agent is selected from the group consisting of daunorubicin, doxorubicin, epirubicin, idarubicin, pixantrone, sabarubicin, and valrubicin.

9. A composition for treating cancer comprising the recombinant exosome of claim 1 as an active ingredient.

10. A composition for treating cancer comprising the recombinant exosome of claim 5 as an active ingredient.

11. The composition of claim 9, wherein the anticancer compound is an immunogenic cell death agent, an immune checkpoint inhibitor, amitotic inhibitor, an antimetabolite, a hormone, an alkylating agent, a Flt3 ligand or a topoisomerase inhibitor.

12. The composition of claim 11, wherein the immunogenic cell death-inducing chemotherapeutic agent is selected from the group consisting of anthracycline-based anticancer agents, cetuximab, taxane-based anticancer agents, bleomycin, cyclophosphamide, cardiac glycosides, GADD34/PP1 inhibitors, mitoxantrone and oxaliplatin.

13. The compound of claim 12, wherein the anthracycline-based anticancer agent is selected from the group consisting of daunorubicin, doxorubicin, epirubicin, idarubicin, pixantrone, sabarubicin, and valrubicin.

14. The composition of claim 12, wherein said cardiac glycoside is used in combination with a non-immunogenic apoptosis-inducing chemotherapeutic agent.

15. The composition of claim 12, wherein said GADD34/PP1 inhibitor is used in combination with mitomycin.

16. The composition of claim 12, wherein the taxane-based anticancer agent is paclitaxel or docetaxel.

17. The composition of claim 11, wherein the immune checkpoint inhibitor is a PD-1/PD-L1 interaction inhibitor or a CTLA-4/B7-1/B7-2 interaction inhibitor.

18. The composition of claim 17, Wherein the PD-1/PD-L1 interaction inhibitor is Pembrolizumab, Nivolumab, Atezolizumab or Avelumab.

19. The method of claim 17, wherein the CTLA-4/B7-1/B7-2 interaction inhibitor is Ipilimumab.

20. A composition for treating cancer comprising a recombinant exosome of claim 1 and an anticancer compound as active ingredient.

21. The composition of claim 20, wherein the anticancer compound is an immunogenic cell death-inducing chemotherapeutic agent, an immune checkpoint inhibitor, amitotic inhibitor, an antimetabolite, a hormone, an alkylating agent, a Flt3 ligand or a topoisomerase inhibitor.

22. The composition of claim 21, wherein the immunogenic cell death-inducing chemotherapeutic agent is selected from the group consisting of anthracycline-based anticancer agent, cetuximab, taxane-based anticancer agent, bleomycin, cyclophosphamide, cardiac glycosides, GADD34/PP1 inhibitors, mitoxantrone and oxaliplatin.

23. The compound of claim 22, wherein the anthracycline-based anticancer agent is selected from the group consisting of daunorubicin, doxorubicin, epirubicin, idarubicin, pixantrone, sabarubicin, and valrubicin.

24. The method of claim 22, wherein the cardiac glycosides are used in combination with a non-immunogenic apoptosis-inducing chemotherapeutic agent.

25. The method of claim 22, wherein the GADD34/PP1 inhibitor is used in combination with mitomycin.

26. The method of claim 22, wherein the taxane-based anticancer agent is paclitaxel or docetaxel.

27. The composition of claim 21, wherein the immune checkpoint inhibitor is a PD-1/PD-L1 interaction inhibitor or a CTLA-4/B7-1/B7-2 interaction inhibitor.

28. The composition of claim 27, wherein the PD-1/PD-L1 interaction inhibitor is Pembrolizumab, Nivolumab, Atezolizumab or Avelumab.

29. The method of claim 27, wherein the CTLA-4/B7-1/B7-2 interaction inhibitor is Ipilimumab.

30. A method for treating a subject suffering from cancer comprising administering the recombinant exosome of claim 1 to the subject.

31. The method of treating cancer according to claim 30, wherein an anticancer compound is further administered to the subject.

32. The method according to claim 31, wherein the anticancer compound is administered simultaneously or sequentially with the recombinant exosome.

33. The method of claim 30, further comprising applying photodynamic therapy or radiation therapy to the subject.

34. A method for treating a subject suffering from cancer, comprising administering a therapeutically effective amount of recombinant exosome of claim 5 to the subject.

35. The method of claim 24, further comprising applying photodynamic therapy or radiation therapy to the subject.


SEQUENCE LISTING (30 sequences)

SEQ ID NO: 1 - protein, 455 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 2 - protein, 455 aa, Homo sapiens
LNFRAPPVIPNVAFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 3 - protein, 455 aa, Pan troglodytes
LNFRAPPVIPNVPFLWAWNAPSEFCLGKFDEPLDMSLFSFIGSPRINVTGQDVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVQEAIRVSKIPDAKSPLPVFVYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEESQIFYNASPSTLS

SEQ ID NO: 4 - protein, 455 aa, Pan paniscus
LNFRAPPVIPNVPFLWAWNAPSEFCLGKFDEPLDMSLFSFIGSPRINVTGQDVTIFYVDRLGYYPYIDSITGVTVNGGIPQKISLQDHLDKAKKDITFYMPVDNLGMAVIDWEEWRPTWARNWKPKDIYKNRSIELVQQQNVQLNLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFVYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEESQIFYNASPSTLS

SEQ ID NO: 5 - protein, 455 aa, Gorilla gorilla
LNFRAPPVIPNVPFLWAWNAPSKFCLGKFDEPLDMSLFSLIGSPRINITGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFMVETIKLGKLLRPNHLWGYYLFPDCYNHHYKKPGYNGSCFNVEIKRNDDLSWLWNESTALYPSIYLNTQQSPVAATLYVRNRVQEAIRVSKIPDAKSPLPVFVYTRIVFTDQVLKFLSQDELVYTFGETVALGASGIVIWGTLSIMRSMKSCLLLDNYMETILNPYIINVTLAAKMCSQVLCQEQGVCIRKDWNSSDYLHLNPDNFAIQLEKGGKFTVRGKPTLEDLEQFSEKFYCSCYSTLSCKEKADIKDTDAVDVCIADGVCIDAFLKPPMDTEESQIFYNASPSTLS

SEQ ID NO: 6 - protein, 455 aa, Pan troglodytes
LNFRAPPVIPNVPFLWAWNAPSEFCLGKFDEPLDTSLFSFIGSPRINVTGQDVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYARNRVQEAIRVSKIPDAKSPLPVFVYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVYIDAFLKPPMETEESQIFYNASPSTLS

SEQ ID NO: 7 - protein, 455 aa, Gorilla gorilla; identical to SEQ ID NO: 5

SEQ ID NO: 8 - protein, 455 aa, Pongo abelii
LNFRAPPIIPNMPFLWAWNAPSEFCLGKFDEPLDMSLFSLIGSPRINVTGQAVTIFYVDRLGYYPYIDSITGVTVNGGIPQKISLQDHLDKAKKDILFYMPVDNLGMAVIDWEEWRPTWARNWKPKDVYKNRSIELVQQQNVQLNLTEATEKAKQEFEKAGKDFMVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFVYARIVFTDQVLKFLSQDELVYTFGETVALGASGIVIWGSLSIMRSMKSCLLLDNYMETILNPYIINVTLAAKMCSQVLCQEQGVCIRKDWNSSDYLHLNPDNFAIQLEKGGKFTVRGKPTLEDLEQFSEKFYCSCYSTLSCKEKADVKDTDAVDVCIADGVCIDAFLKPPMETEESQIFYNASPSTLS

SEQ ID NO: 9 - protein, 455 aa, Pongo abelii; identical to SEQ ID NO: 8

SEQ ID NO: 10 - protein, 455 aa, Hylobates lar
LNFRAPPVIPNVPFLWAWNAPSEFCLGKFDEPLDMSLFSLIGSPRINVTGQGVTIFYVDRLGYYPYIDSITGVTVNGGIPQKISLQDHLDKAKQDITFYMPVDNLGMAVIDWEEWRPTWARNWKPKDVYKNRSIELVQQQNVQLSLAEATEKAKQEFEKAGKDFMVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFVYARIVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQLEKGGKFTVRGKPTPEDLEQFSEKFYCSCYSTLSCKEKADVKDTDAVDVCIADGVCIDAFLKPPKETEESQIFYNASPSTLS

SEQ ID NO: 11 - protein, 455 aa, Symphalangus syndactylus
LNFRAPPVIPNVPFLWAWNAPSEFCLGKFDEPLDMSLFSLIGSPRINVTGQGVTIFYVDRLGYYPYIDSITGVTVNGGIPQKISLQDHLDKAKQDITFYMPVDNLGMAVIDWEEWRPTWARNWKPKDVYKNRSIELVQQQNVQLNLTEATEKAKQEFEKAGKDFMVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFVYARIVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQLEKGGKFTVRGKPTPEDLEQFSEKFYCSCYSTLSCKEKADVKDTDAVDVCIADGVCIDAFLKPPKETEESQIFYNASPSTLS

SEQ ID NO: 12 - protein, 455 aa, Nomascus leucogenys
LNFRAPPVIPNVPFLWAWNAPSEFCLGKFDEPLDMSLFSLTGSPRINVTGQGVTIFYVDRLGYYPYIDSITGVTVNGGIPQKISLQDHLDKAKQDITFYMPVDNLGMAVIDWEEWRPTWARNWKPKDVYKNRSIELVQQQNVQLSLAEATEKAKQEFEKAGKDFMVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFVYARIVFTDQVLKFLSRDELVYTLGETVALGASGIVIWGSLSIVRSMKSCLLLDNYMETILNPYIINVTLAAKMCSQVLCQEQGVCIRKDWNSSDYLHLNPDNFAIQLEKGGKFTVRGKPTPEDLEQFSEKFYCSCYSTLSCKEKADVKDTDAVDVCIADGVCIDAFLKPPKETEESQIFYNASPSTLS

SEQ ID NO: 13 - protein, 455 aa, Nomascus leucogenys; identical to SEQ ID NO: 12

SEQ ID NO: 14 - protein, 456 aa, Nasalis larvatus
LNFRAPPIIPNVPFLWAWNAPSEFCLGKFDEPLDMSLFSLIGSPRINVTGQGVTIFYVDRLGYYPYIDITTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMLETIKLGKSLRPNHLWGYYLFPDCYNHHYKKPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTLEDLEEFSEKFYCSCYTTLSCKEKADVKDTDAVDVCIADGVCIDASLKPPVETEGSPPIFYNTSPSTLS

SEQ ID NO: 15 - protein, 456 aa, Colobus angolensis
LNFRAPPVIPNVPFLWAWNAPSEFCLGKFDEPLDMSLFSLIGSPRINVTGQGVTIFYVDRLGYYPYIDVTTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMLETIKLGKSLRPNHLWGYYLFPDCYNHHYKRPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTPEDLEEFSEKFYCSCYTTLSCKEKADVKDTDAVDVCIADGVCIDASLKPPVETEGSPPIFYNASPSTLS

SEQ ID NO: 16 - protein, 456 aa, Colobus guereza
LNFRAPPIIPNVPFLWAWNAPSEFCLGKFDEPLDMSLFSLIGSPRINVTGQGVTIFYVDRLGYYPYIDVTTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMLETIKLGKSLRPNHLWGYYLFPDCYNHHYKRPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTPEDLEEFSEKFYCSCYTTLSCKEKADVKDTDAVDVCIADGVCIDASLKPPVETEGSPPIFYNASPSTLS

SEQ ID NO: 17 - protein, 456 aa, Semnopithecus entellus
LNFRAPPIIPNVPFLWAWNAPSEFCLGKFDEPLDMSLFSLIGSPRINVTGQGVTIFYVDRLGYYPYIDITTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMIETIKLGKSLRPNHLWGYYLFPDCYNHHYKKPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTPEDLEEFSEKFYCSCYTTLSCKEKTDVKDTDAVDVCIADGVCIDASLKPPVETEGSPPIFYNTSPSTLS

SEQ ID NO: 18 - protein, 456 aa, Colobus angolensis; identical to SEQ ID NO: 15

SEQ ID NO: 19 - protein, 456 aa, Pygathrix nemaeus
  VARIANT (412): Xaa is unidentified amino acid
LNFRAPPIIPNVPFLWAWNAPSEFCLGKFDEPLDMSLFSLIGSPRINVTGQGVTIFYVDRLGYYPYIDITTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMLETIKLGKSLRPNHLWGYYLFPDCYNHHYKKPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTPEDLEEFSEKFYCSCYTTLSCKEKXDVKDTDAVDVCIADGVCIDASLKPPVETEGSPPIFYNTSPSTLS

SEQ ID NO: 20 - protein, 456 aa, Rhinopithecus roxellana
LNFRAPPIIPNVPFLWAWNAPSEFCLGKFDEPLDMSLFSLIGSPRINVTRQGVTIFYVDRLGYYPYIDITTGVTVHGGIPQKVSLQDHLDKAKQDIIFYMPVDNLGMAVIDWEEWRPTWARNWKPKDVYKNRSIELVQQQNVQLSLPEATDKAKQEFEKAGKDFMLETIKLGKSLRPNHLWGYYLFPDCYNHHYKKPGYNGSCFDVEIKRNDDLSWLWNESTALYPSIYLNTQQSPVAATLYVRNRVQEAIRVSKIPDAKNPLPVFVYARLVFTDQVLKFLSRDELVYTLGETVALGASGIVIWGSLSIMRSMKSCLLLDNYMETILNPYIINVTLAAKMCSQVLCQEQGVCIRKDWNSSDYLHLNPDNFAIQMEKGGKFTVRGKPTPEDLEEFSEKFYCSCYTTLSCKEKADVKDTDAVDVCIADGVCIDASLKPPVETEGSPPIFYNTSPSTLS

SEQ ID NO: 21 - protein, 456 aa, Saguinus oedipus
LNFRAPPVIPNVPFLWAWNAPSEFCLGKFDEPLDMSLFSLIGSPRINVTGQGVTIFYVDRLGYYPYIDPTTGVIVNEGIPQKVSLQDHLDKARKDIIFYMPVDNLGMGVIDWEEWRPTWARNWKPKDIYKNKSIEMVQQRNVQLNLTQATDIAKQEFEKAAKDFMLETIKLGKALRPNHLWGYYLFPDCYNHHYKKPGYNGSCFDIEIKRNDDLSWLWNESTALYPSIYLNTQQSAVAAVLYVRNRVQEAIRVSKIPNANSPLPVFVYARLVFTDQVLRFLSQDELVYTLGETVALGASGIVIWGSLSIMRSMKSCLLLDTYMETVLNPYIINTTLAAKMCSQVLCQEQGVCIRKDWNSSDYLHLNPDNFAIEIEKGGKFTVRGKPTFEDLEQFSEKFYCSCYTTLSCKAKADVKDTDAVDVCIADGVCIDASLKPPKETEESSQIFYNPSSSTPS

SEQ ID NO: 22 - protein, 456 aa, Cercopithecus mitis
LNFRAPPIIPNVPFLWAWNAPSEFCLGKFDEPLDMSLFTLIGSPRINVTGQGVTIFYVDRLGYYPYIDLTTGDIVHGGIPQKVSLQDHLDKSKQDIIFYMPVDNLGMAVIDWEEWRPTWARNWKPKDVYRNRSIELVQQQNVQLSLPQATDKAKQEFEKAGKDFMLETIKLGRSLRPNHLWGYYLFPDCYNHHYKKPGYNGSCLDVEMKRNDDLSWLWNESTALYPSIYLNTQQSLVVATLYVRNRVREAIRVSKIPDAKNPLPVFVYARLVFTDQVLKFLSREELVSTLGETVALGASGIVIWGSLSIMRSMKSCLLLDTYMETILNPYIINVTLAAKMCSQVLCQEQGVCIRKDWNSSDYLHLNPDNFAIQLEKGGKFTVRGKPTVEDLEEFSEKFYCSCYTNLSCKEKADVKDTDAVDVCIADGVCIDASLKPPVETEGSPPIFYNTSSCTVS

SEQ ID NO: 23 - protein, 456 aa, Macaca nemestrina
LNFRAPPIIPNVPFLWAWNAPSEFCLGKFDEPLDMSLFTLMGSPRINVTGQGVTIFYVDRLGYYPYIDLTTGVTVHGGIPQKVSLQDHLDKSKQDILFYMPVDNLGMAVIDWEEWRPTWARNWKPKDVYKNRSIELVQQQNVQLSLPQATDKAKQEFEKAGKDFMLETIKLGRSLRPNHLWGYYLFPDCYNHHYRKPGYNGSCFDVEIKRNDDLSWLWNESTALYPSIYLNTQQSVVVATLYVRNRVREAIRVSKIPDAKNPLPVFVYARLVFTDQVLKFLSREELVSTLGETVALGASGIVIWGSLSITRSMKSCLLLDTYMETILNPYIINVTLAAKMCSQVLCQEQGVCIRKDWNSSDYLHLNPDNFDIRLEKGGKFTVHGKPTVEDLEEFSEKFYCSCYTNLSCKEKADVKDTDAVDVCIADGVCIDASLKPPVETEGSPPIFYNPSSSTVS

SEQ ID NO: 24 - protein, 456 aa, Aotus nancymaae
LNFRAPPIIPNVPFLWAWNAPSESCLGKFDEPLDMSLFSLIGSPRINITGQGVTIFYVDRLGYYPYIDPTTGVIMNGGIPQKMPLQDHLDKARKDVTFYMPVDNLGMGVIDWEEWRPTWARNWKPKDIYKNKSIEMVQQRNVQLNLTQATDIAKQEFEKAAKDFMLETIKLGKALRPNHLWGYYLFPDCYNHHYKKPGYNGSCFNVEIKRNDDLSWLWNESTALYPSIYLNTQQSAIAAMLYVRNRVQEAIRVSKIPDANSPLPVFVYARLVFTDQVLRFLSQDELVYTLGETVALGASGIVIWGSLSIMRSMKSCLLLDTYMETVLNPYIINTTLAAKMCSQVLCQEQGVCIRKDWNSSDYLHLNPDNFAIEIEKGGKFTVHGKPTFEDLEQFSEKFYCSCYTTLSCKAKADVKDTDAVDVCIADGVCIDASLKPPKETEESSQIFYNPSSSTPS

SEQ ID NO: 25 - protein, 456 aa, Saguinus fuscicollis
  VARIANT (311): Xaa is unidentified amino acid
LNFRAPPIIPNVPFLWAWNAPSEFCLGKFDEPLDMSLFSLIGSPRINVTGQGVTIFYVDRLGYYPYIDPTTGVIVNEGIPQKISLQDHLDKARKDIIFYMPVDNLGMGVIDWEEWRPTWARNWKPKDIYKNKSIEMVQQRNVQLNLTQATDIAKQEFEKAAKDFMLETIKLGKALRPNHLWGYYLFPDCYNHHYKKPGYNGSCFDIEIKRNDDLSWLWNESTALYPSIYLNTQQSAVAAVLYVRNRVQEAIRVSKIPNANSPLPVFVYARLVFTDQVLRFLSQDELVYTLGETVALGASGIVIWGSLSIMXSMKSCLLLDTYMETVLNPYIINTTLAAKMCSQVLCQEQGVCIRKDWNSSDYLHLNPDNFAIEIEKGGKFTVRGKPTFEDLEQFSEKFYCSCYTTLSCKAKADVKDTDAVDVCIADGVCIDASLKPPKETEESSQIFYNPSSSTPS

SEQ ID NO: 26 - protein, 456 aa, Callimico goeldii
LNFRAPPVIPNVPFLWAWNAPSEFCLGKFDEPLDMSLFSLIGSPRINVTGQGVTIFYVDRLGYYPYIDPTTGVIVNEGIPQKIALQDHLDKARKDIIFYMPIDNLGMGVIDWEEWRPTWARNWKPKDIYKNKSIEMVQQQMVQLNLTQATDIAKQEFEKAAKDFMLETIKLAKTLRPNHLWGYYLFPDCYNHHYKKPGYNGSCFDIEIKRNDDLSWLWNESTALYPSIYLNTQQSAVAAVLYVRNRVQEAIRVSKIPNANSPLPVFVYARLVFTDQVLRFLSQDELVYTLGETVALGASGIVIWGSLSIMRSMKSCLLLDTYMETVLSPYIINTTLAAKMCSQVLCQEQGVCIRKDWNSSDYLHLNPDNFAIEIEKGGKFTVRGKPTFEDLEQFSEKFYCSCYSTLSCKAKADVKDTDAVDVCIADGVCIDASLKPPKETEESSQIFYNPSSSIPS

SEQ ID NO: 27 - protein, 456 aa, Erythrocebus patas
LNFRAPPIIPNVPFLWAWNAPSEFCLGKFNEPLDMSLFTLIGSPRINVTGQGVTIFYVDRLGYYPYIDLTTGVTVHGGIPQKVSLQDHLDKSKQDIIFYMPVDNLGMAVIDWEEWRPTWARNWKPKDVYKNRSIELVQQQNVQLSLPQATDKAKQEFEKAGKDFMLETIKLGRSLRPNHLWGYYLFPDCYNHHYKKPDYNGSCFDVEIKRNDDLSWLWNESTALYPSIYLNSQQSLVVATLYVRNRVREAIRVSKIPDAKNPLPVFVYARLVFTDQVLKFLSREELVSTLGETVALGASGIVIWGSLSIMRSMKSCVLLDNYMETTLNPYIINVTLAAKMCSQVLCQEQGVCIRKDWNSSDYLHLNPDNFVIQLEKGGKFTVHGKPTVEDLEEFSEKFYCSCYTNLSCKEKADVKDTDAVDVCIADGVCIDASLKPPVETEGSPPIFYNTSSSTVS

SEQ ID NO: 28 - protein, 456 aa, Chlorocebus aethiops
LNFRAPPIIPNVPFLWAWNAPSEFCLGQFNEPLDMSLFTLIGSPRINVTGQGVTIFYVDRLGYYPYIDLTTGVTVHGGIPQKVSLQDHLDKSKQDIIFYMPVDNLGMAVIDWEEWRPTWARNWKPKDVYKNRSIELVQQQNVQLSLPQATDKAKQEFEKAGKDFMLETIKLGRSLRPNHLWGYYLFPDCYNHHYKKPGYNGSCFDVEIKRNDDLSWLWNESTALYPSIYLNTQQSLVVATLYVRNRVREAIRVSKIPDAKNPLPVFVYARLVFTDQVLKFLSREELVSTLGETVALGASGIVIWGSLGIMRSMKSCVLLDNYMETTLNPYIINVTLAAKMCSQVLCQEQGVCIRKDWNSSDYLHLNPDNFVIQLEKGGKFTVHGKPTVEDLEEFSEKFYCSCYTNLSCKEKADVKDTDAVDVCIADGVCIDASLKPPVETEGSPPIFYNTSSSTVS

SEQ ID NO: 29 - protein, 456 aa, Papio hamadryas
LNFRAPPIIPNVPFLWAWNAPSEFCLGKFDEPLDMSLFTLMGSPRINVTGQGVTIFYVDRLGYYPYIDLTTGVTVHGGIPQKVSLQDHLDKSKQDILFYMPVDNLGMAVIDWEEWRPTWARNWKPKDVYRNRSIELVQQQNVQLSLPQATDKAKQEFEKAGKDFMLETIKLGRSLRPNHLWGYYLFPDCYNHHYRKPGYNGSCFDVEIKRNDDLSWLWNESTALYPSIYLNTQQSVVVATLYVRNRVREAIRVSKIPDAKNPLPVFVYARLVFTDQVLKFLSREELVSTLGETVALGASGIVIWGSLSIMRSMKSCLLLDTYMETILNPYIINVTLAAKMCSQVLCQEQGVCIRKDWNSSDYLHLNPDNFDIRLEKGGKFTVHGKPTVEDLEEFSEKFYCSCYTNLSCKEKVDVKDTDAVDVCIADGVCIDASLKPPVETEGSPPIFYNTSSSTVS

SEQ ID NO: 30 - protein, 456 aa, Chlorocebus sabaeus
LNFRAPPIIPNVPFLWAWNAPSEFCLGQFNEPLDMSLFTLTGSPRINVTGQGVTIFYVDRLGYYPYIDLTTGVTVHGGIPQKVSLQDHLDKSKQDIIFYMPVDNLGMAVIDWEEWRPTWARNWKPKDVYKNRSIELVQQQNVQLSLPQATDKAKQEFEKAGKDFMLETIKLGRSLRPNHLWGYYLFPDCYNHHYKKPGYNGSCFDVEIKRNDDLSWLWNESTALYPSIYLNTQQSLVVATLYVRNRVQEAIRVSKIPDAKNPLPVFVYARLVFTDQVLKFLSREELVSTLGETVALGASGIVIWGSLSIMRSMKSCVLLDNYMETTLNPYIINVTLAAKMCSQVLCQEQGVCIRKDWNSSDYLHLNPDNFVIQLEKGGKFTVHGKPTVEDLEEFSEKFYCSCYTNLSCKEKADVKDTDAVDVCIADGVCIDASLKPPVETEGSPPIFYNTSSSTVS